(19) United States
(12) Patent Application Publication
(10) Pub. No.: US 2024/0141447 A1
(43) Pub. Date: May 2, 2024

(54) Dynamic Clinical Assay Pipeline for Detecting a Virus

(71) Applicant: Laboratory Corporation of America Holdings, Burlington, NC (US)

(72) Inventors: Jonathan David Williams, Hillsborough, NC (US); Lakshmanan Krishnan Iyer, Franklin, MA (US)

(21) Appl. No.: 18/500,064

(22) Filed: Nov. 1, 2023

Related U.S. Application Data
(60) Provisional application No. 63/591,611, filed on Oct. 19, 2023.
(60) Provisional application No. 63/491,652, filed on Mar. 22, 2023.
(60) Provisional application No. 63/421,345, filed on Nov. 1, 2022.

(51) Int. Cl. C12Q 1/70; G16B 20/00; G16B 30/10

(52) U.S. Cl. CPC C12Q 1/701; G16B 20/00; G16B 30/10

(57) ABSTRACT

Disclosed herein are methods and systems comprising obtaining nucleic acid from a sample that was obtained from a subject; capturing and amplifying a target molecule in the nucleic acid using a molecular inversion probe under hybridization conditions; ligating an adapter to create a circular molecule; sequencing the circular molecule to obtain sequence reads; generating a sequencing file comprising the sequence reads of each molecule and a position of each sequence read in a reference genome of a virus; and generating a reporting file for the subject comprising a predicted lineage of the virus in the sample.

### CROSS REFERENCE TO RELATED APPLICATIONS

The present application claims priority and benefit from U.S. Provisional Application No. 63/591,611, filed on Oct. 19, 2023, U.S. Provisional Application No. 63/491,652, filed on Mar. 22, 2023, and U.S. Provisional Application No. 63/421,345, filed on Nov. 1, 2022, the entire contents of which are incorporated herein by reference for all purposes.

### FIELD

The present disclosure relates to clinical testing for a virus, and in particular to a dynamic clinical assay pipeline for detecting a virus (e.g., an Orthopoxvirus such as the Monkeypox virus) in a sample and/or assigning a lineage to the virus.

### BACKGROUND

A virus is an infectious microscopic agent that can replicate itself in the living cells of an organism. Although some viruses are harmless to humans, a variety of viruses can cause a wide range of diseases and illnesses, including those that are life-threatening, to animals, plants, microorganisms, and human beings. Understanding the nature of viruses, especially their genetic materials, and detecting their presence is essential for developing effective treatments and clinical protocols as well as preventing the spread of the viral infections.

Traditional virus detection methods include diagnostic tests. However, virus detection may also be hindered by the lack of specific and sensitive diagnostic tests. Many viral infections share similar symptoms, and it can be difficult to distinguish between different viruses based on symptoms alone. Diagnostic tests that are highly specific and sensitive are therefore essential for accurate virus detection but developing such tests can be a time-consuming and complex process.

The replication of a virus relies on its genetic materials. With the development of laboratory technology, computing technology and sequencing technology, machines including sequencers and associated software have been invented and manufactured to identify the presence of a virus in a biological sample such as blood, saliva, or tissue based on the genetic materials in the sample. Different methods, such as Polymerase Chain Reaction (PCR) and Next-generation sequencing (NGS), have also been developed to facilitate the detection of viruses. However, the detection of nucleic acid of a virus in a sample can still be challenging, partially for the reason that viruses are mutated rapidly. The mutation of a virus can result in new lineages that cause different immune responses of their host organism and may need different treatments and protocols to prevent its spread or infection. However, there is lacking a reliable method or system to detect a virus in a sample and assign a lineage to the virus in the sample.

### SUMMARY

In various embodiments, a method is provided that includes: obtaining nucleic acid from a sample that was obtained from a subject; capturing a target molecule in the nucleic acid using a molecular inversion probe under hybridization conditions; amplifying the target molecule using polymerase chain reaction (PCR) to obtain a plurality of amplified molecules; for each molecule in the plurality of amplified molecules, ligating an adapter to each end of the molecule to create a circular molecule; and sequencing the circular molecule to obtain sequence reads; generating, using a computing system, a sequencing file comprising the sequence reads of each molecule in the plurality of amplified molecules and a position of each sequence read in a reference genome of a virus by aligning the sequence read to the reference genome of the virus; and generating, using the computing system and the sequencing file, a reporting file for the subject, wherein the reporting file comprises a predicted lineage of the virus in the sample, wherein the generating comprises: generating a consensus sequence for the target molecule based on sequence reads of each molecule in the plurality of amplified molecules, wherein a nucleotide identity is assigned to a position in the consensus sequence if at least a predetermined number of the sequence reads has the nucleotide identity in the position, and wherein an “N” is assigned to a position in the consensus sequence if less than a predetermined number of the sequence reads has the nucleotide identity in the position; determining one or more scores of the consensus sequence based on the reference genome of the virus or a library of the virus, wherein the one or more scores are determined based on a distribution of mutations of the virus; and determining the predicted lineage of the virus in the sample based on the one or more scores of the consensus sequence.

In some embodiments, the reporting file further comprises a presence or absence of the virus in the sample.

In some embodiments, the presence or absence of the virus in the sample is determined using real-time PCR (RT-PCR) or based on the one or more scores of the consensus sequence.

In some embodiments, the subject is tested positive regarding the virus and the sample comprises vial nucleic acid.

In some embodiments, the virus is monkeypox (MPX) virus.

In some embodiments, the target molecule is a double-stranded DNA molecule.

In some embodiments, the molecular inversion probe consists of two binding sites about 600-700 bp apart.

In some embodiments, the sequencing file is a Binary Alignment Map (BAM) file.

In some embodiments, the reporting file is a Variant Call Format (VCF) file.

In some embodiments, the method further comprises providing a treatment plan or clinical testing protocol for the subject.

In some embodiments, the treatment plan comprises administering antiviral medications for the subject.

In some embodiments, the method further comprises obtaining a prevalent lineage of the virus for a subject population, wherein the prevalent lineage is determined based on the predicted lineage of the virus in the sample; updating the molecular inversion probes to capture the target molecule, wherein the target molecule is specific to the prevalent lineage; updating the adapters based on the updated molecular inversion probe; and obtaining a set of decision rules that are specific to determine the prevalent lineage, wherein the determining the one or more scores of the consensus sequence is determined using the set of decision rules.

In various embodiments, a method is provided that includes: obtaining nucleic acid from a sample that was obtained from a subject; capturing a plurality of target molecules in the nucleic acid using a plurality of molecular inversion probes under hybridization conditions; amplifying each target molecule using polymerase chain reaction (PCR) to obtain a plurality of sets of amplified molecules, wherein each set of amplified molecules corresponds to a target molecule; for each molecule in each set of amplified molecules, ligating an adapter to each end of the molecule to create a circular molecule; and sequencing the circular molecule to obtain sequence reads; generating, using a computing system, a sequencing file comprising the sequence reads of each molecule in each set of amplified molecules and a position of each sequence read in a reference genome of a virus by aligning the sequence read to the reference genome of the virus; and generating, using the computing system and the sequencing file, a reporting file for the subject, wherein the reporting file comprises a predicted lineage of the virus in the sample, wherein the generating comprises: generating a consensus sequence for each target molecule based on sequence reads of each molecule in the set of amplified molecules corresponding to the target molecule, wherein a nucleotide identity is assigned to a position in the consensus sequence if at least a first predetermined number of the sequence reads has the nucleotide identity in the position, and wherein an “N” is assigned to a position in the consensus sequence if less than the first predetermined number of the sequence reads has the nucleotide identity in the position; generating a genome construct for the nucleic acid in the sample based on the consensus sequences for the target molecules, wherein a nucleotide identity is assigned to a position in the genome construct if at least a second predetermined number of the consensus sequences has the nucleotide identity in the position and the nucleotide identity in the position is present in at least 50% of the sequence reads that aligned to the position, and wherein an “N” is assigned to a position in the genome construct if less than the second predetermined number of the consensus sequences has the nucleotide identity in the position; determining one or more scores of the consensus sequence based on the reference genome of the virus or a library of the virus; and determining the predicted lineage of the virus in the sample based on the one or more scores of the consensus sequence.

In some embodiments, the reporting file further comprises a presence or absence of the virus in the sample.

In some embodiments, the presence or absence of the virus in the sample is determined using real-time PCR (RT-PCR) or based on the one or more scores of the consensus sequence.

In some embodiments, the subject is tested positive regarding the virus and the sample comprises vial nucleic acid.

In some embodiments, the virus is monkeypox (MPX) virus.

In some embodiments, the target molecule is a double-stranded DNA molecule.

In some embodiments, the molecular inversion probe consists of two binding sites about 600-700 bp apart.

In some embodiments, the sequencing file is a Binary Alignment Map (BAM) file.

In some embodiments, the reporting file is a Variant Call Format (VCF) file.

In some embodiments, the method further comprises providing a treatment plan or clinical testing protocol for the subject.

In some embodiments, the treatment plan comprises administering antiviral medications for the subject.

In some embodiments, the method further comprises obtaining a prevalent lineage of the virus for a subject population, wherein the prevalent lineage is determined based on the predicted lineage of the virus in the sample; updating the plurality of molecular inversion probes to capture mutations that are specific to the prevalent lineage; updating the adapters based on the updated plurality of molecular inversion probes; and obtaining a set of decision rules that are specific to determine the prevalent lineage, wherein the determining the one or more scores of the consensus sequence is determined using the set of decision rules.

In some embodiments, a system is provided that includes one or more data processors and a non-transitory computer readable medium containing instructions which, when executed on the one or more data processors, cause the one or more data processors to perform part or all of one or more methods or processes disclosed herein.

In some embodiments, a computer-program product is provided that is tangibly embodied in a non-transitory machine-readable medium and that includes instructions configured to cause one or more data processors to perform part or all of one or more methods disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the techniques claimed. Thus, it should be understood that although the present techniques have been specifically disclosed by embodiments and optional features, modification and variation of the concepts herein disclosed may be resorted to by those skilled in the art, and that such modifications and variations are considered to be within the scope of the techniques as defined by the appended claims.

### BRIEF DESCRIPTION OF THE DRAWINGS

The present invention will be better understood in view of the following non-limiting figures, in which:

FIG. 1 is a block diagram of a system that is capable of performing virus detection and lineage assignment techniques in accordance with various embodiments.

FIG. 2 is an exemplary flowchart showing a process for generating a reporting file concerning a presence or absence of a virus in a sample in accordance with various embodiments.

FIG. 3 is an exemplary flowchart showing a process for performing disclosed techniques concerning a presence or absence of a virus in a sample in accordance with various embodiments.

FIG. 4 illustrates a portion of a Molecular Loop Probe Set Design and PCR Pipeline for capturing target DNA molecules in accordance with various embodiments.

FIG. 5 illustrates a portion of an Adapter Design, Ligation, and Sequencing Pipeline for generating circular DNA molecules in accordance with various embodiments.

FIG. 6 shows an overview of the complexities of the MPX genome in accordance with various embodiments.

FIG. 7 is an Integrative Genomics Viewer (IGV) plot illustrating genome coverage in accordance with various embodiments.

FIG. 8 is a higher resolution graphical representation of the IGV plot shown in FIG. 7 generated using software developed for various clinical samples in accordance with various embodiments.

FIG. 9 illustrates an alternative way to demonstrate the coverage of the genome and read depth of various samples and controls in accordance with various embodiments.

FIG. 10 illustrates genome coverage and per-base coverage in accordance with various embodiments.

FIG. 11 illustrates exemplary virus calls and lineage assignments in samples in accordance with various embodiments.

FIGS. 12A and 12B illustrate a lineage analysis of initial pilot run showing expected assignments for controls and circulating strains patient samples in accordance with various embodiments.

FIGS. 13A-13C illustrates an analysis of larger multiplex run containing more control and patient samples in accordance with various embodiments.

FIG. 14 demonstrates a comparison between the sequenced synthetic control and the published data in accordance with various embodiments.

FIG. 15 illustrates a Molecular Loop Sequencing process in accordance with various embodiments.

FIG. 16 illustrates an example computing device suitable for use with systems and methods for determining a presence or absence of a virus and its predicted lineage in a sample obtained from a subject in accordance with various embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart or diagram may describe the operations as a sequential process, many of the operations may be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

### I. Introduction

Viruses can be life-threatening and there is a need to be able to accurately detect viruses from a biological sample, identify different viral strains, and assign lineages. Because the replication of a virus relies on its genetic materials, for example, its nucleic acid, various methods have been developed to detect a virus in a sample based on the nucleic acid from the sample. The detection and confirmation of nucleic acid of a virus in a biological sample can be challenging. One of the biggest reasons is that viruses are mutated rapidly, with an average mutation rate of 10⁻⁴ to 10⁻⁸ mutations per replication site. The mutation changes the genetic materials of the virus, alters its surface proteins and other important components, and affects host organism's immune responses as well as results of a diagnostic test.

Another challenge associated with virus detection is the existence of long stretches of repetitive DNA or RNA. Sequencing is often involved in the detection of viruses for accurately identifying different viral strains, monitoring primer sites, as well as assigning lineages. However, without accurate sequencing depth and coverage, repetitive sequences in the nucleic acid of viruses may be difficult to be distinguished from host nucleic acid samples as well as mask the presence of mutations or variants and lead to false negative results.

To address these challenges and others, techniques described herein are directed to methods and systems to construct a dynamic clinical assay pipeline that is capable of capturing and analyzing nucleic acid of a virus to provide accurate virus detection and lineage assignment results. The dynamic clinical assay pipeline can be dynamically modified based on factors such as prevalence of viruses and/or a historical lineage assignment to provide more efficient and accurate virus detection. In particular, molecular inversion probes may be designed, manufactured, and used to capture target nucleic acid molecules and PCR methods may be used to amplify the captured molecules. To secure adequate sequencing depths and coverage for accurate virus detection and lineage assignment, the amplified molecules can be ligated to adapters with selected size to acquire circular molecules. The circular molecules are sequenced using desirable sequencing methods such as circular consensus sequencing and a consensus sequence for each target nucleic acid molecule is determined based on a predetermined rule. A sequencing file is generated to store information comprising sequence reads and their positions in a reference genome, so that the sequencing file and stored data can be used to adjust and generate a report file. The report file comprises virus detection and/or lineage assignment information that are determined based on the consensus sequences. The virus detection and/or lineage assignment information can be used to dynamically modify the molecular inversion probes and/or adapters to provide a dynamic clinical assay pipeline and provide efficient and accurate virus detection and lineage assignment. A system using the described techniques can achieve a 98.84% accuracy in detecting a virus and assigning a correct lineage without removing uncovered regions or masking for repeats.

There are various advantages associated with and achieved by the techniques described herein. First, wet-lab techniques including the specific designed molecule capture and ligation methods enable the quality of molecules to be sequenced and the sequencing techniques such as circular consensus sequencing double-secure the even read coverage across the virus genome. Second, because more qualified sequence reads per molecule can be obtained and secured, less molecules per sample are needed and more samples can be sequenced together, which increase sequencing efficiency and decrease sequencing costs. Third, the techniques described herein are universally applicable and/or can be readily modified for the detection of different lineages and types of viruses, and thus can be implemented as a dynamic clinical assay pipeline for detecting viruses. Also, as the whole system is specifically designed and the read counts are predictable, the sequencing process can be optimized to guarantee an optimal cost. Last but not least, systems and methods using the described techniques can achieve an improvement in the overall accuracy of the virus detection and lineage assignment.

In various embodiments, a method may comprise: obtaining nucleic acid from a sample that was obtained from a subject; capturing a target molecule in the nucleic acid using a molecular inversion probe under hybridization conditions; amplifying the target molecule using polymerase chain reaction (PCR) to obtain a plurality of amplified molecules; for each molecule in the plurality of amplified molecules, ligating an adapter to each end of the molecule to create a circular molecule; and sequencing the circular molecule to obtain sequence reads; generating, using a computing system, a sequencing file comprising the sequence reads of each molecule in the plurality of amplified molecules and a position of each sequence read in a reference genome of a virus by aligning the sequence read to the reference genome of the virus; generating, using the computing system and the sequencing file, a reporting file for the subject, wherein the reporting file comprises a predicted lineage of the virus in the sample, wherein the generating comprises: generating a consensus sequence for the target molecule based on sequence reads of each molecule in the plurality of amplified molecules, wherein a nucleotide identity is assigned to a position in the consensus sequence if at least a predetermined number of the sequence reads has the nucleotide identity in the position, and wherein an “N” is assigned to a position in the consensus sequence if less than a predetermined number of the sequence reads has the nucleotide identity in the position; determining one or more scores of the consensus sequence based on the reference genome of the virus or a library of the virus; and determining the predicted lineage of the virus in the sample based on the one or more scores of the consensus sequence.

### II. Terms and Definitions

The terminology used herein is for the purpose of describing particular embodiments only and is not intended to be limiting of the disclosure. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer, or section. Thus, a first element, component, region, layer, or section discussed below could be termed a second element, component, region, layer or section without departing from the teachings of the disclosure. The sequence of operations (or steps) is not limited to the order presented in the claims or figures unless specifically indicated otherwise.

As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “from about X to Y” mean “from about X to about Y.”

As used herein, the terms “at home collection” or “self-collection” and the like refer to the use of a kit that may be provided to a subject containing a swab, a container having a transport fluid (e.g., buffer) and a container to return the self-collected sample to a laboratory for testing.

As used herein, the terms “automated” and “automatic” mean that the operations can be carried out with minimal or no manual labor or input. The term “semi-automated” refers to allowing operators some input or activation, but calculations, acquisition, purification, and other steps are done electronically, typically programmatically, without requiring manual input.

As used herein, when an action is “based on” something, this means the action is based at least in part on at least a part of the something.

As used herein, the term “clade” or “lineage” refers to a virus variant or a subset of a virus species, which is defined by genetic differences or its specific combination of mutations or biomarkers.

As used herein, “CT” or “ct” refers to cycle threshold, or the total number of cycles required to amplify and detect a nucleic acid target (e.g., a viral nucleic acid) by real time-PCR and/or PCR.

As used herein, the term “patient” or “subject” is used broadly and refers to an individual that provides a sample for testing or analysis. The individual “patient” or “subject” whereby a sample is collected, obtained, and/or provided by, includes any and all warm-blooded mammalian subjects such as humans and/or animals.

As used herein, the terms “probe,” “probe oligonucleotide,” “oligonucleotide,” and “probe oligonucleotide sequence” can be used interchangeably. The terms “probe,” “probe oligonucleotide,” “oligonucleotide,” and “probe oligonucleotide sequence” may be used to refer to any molecule or system used to detect a target molecule, and the length of a probe or probe oligonucleotide may vary, for example, from 4 nucleotides to about 200 nucleotides.

As used herein, the term “programmatically” means carried out using a computer program and/or software, processor or ASIC directed operations. The term “electronic” and derivatives thereof refer to automated or semi-automated operations carried out using devices with electrical circuits and/or modules rather than via mental steps and typically refers to operations that are carried out programmatically.

As used herein, the term “protocol” refers to an automated electronic algorithm (typically a computer program) with mathematical computations, defined rules for data interrogation and analysis that manipulates a system to perform a set of instructions.

As used herein, repeatability (or intra-assay precision) describes the closeness of agreement between results of successive measurements of the same analyte and carried out under the same conditions of measurement. Intra-assay repeatability is the measurement of the variability when the same specimen is analyzed during one analytical run.

As used herein reproducibility (or inter-assay precision) describes the closeness of agreement between results of successive measurements of the same analyte and carried out under the same conditions of measurement. Inter-assay repeatability is a measurement of the variability when the same specimen is analyzed during more than one run.

As used herein, real-time PCR or quantitative PCR (qPCR) allows for real-time detection of a PCR amplification product. Real-time polymerase chain reaction (PCR) assays use a fluorescent-labeled probe or intercalating dye to visualize a PCR reaction and monitor the quantity of double-stranded DNA product that is produced. The fluorogenic 5′ nuclease assay (i.e., TaqMan® assay) is a real-time PCR assay which uses a fluorogenic probe, consisting of an oligonucleotide with a reporter dye attached to the 5′ end and a quencher dye attached at or near the 3′ end. The probe anneals to a specific target sequence located between the forward and reverse primers. During the extension phase of the PCR cycle, the 5′ nuclease activity of Taq polymerase degrades the probe, causing the reporter dye to separate from the quencher dye and a fluorescent signal is generated. With each cycle, additional reporter dye molecules are cleaved from their respective probes, and the fluorescence intensity is monitored during the PCR. The Taq polymerase used may be inactive at room temperature and activated by incubation at 95° prior to initiating the cycling portion of the assay. This minimizes the production of nonspecific amplification products.

As used herein, the terms “sample,” “patient sample,” “biological sample,” and “specimen” can be used interchangeably. Non-limiting examples of samples that may be used for analysis with the disclosed methods and systems include, blood or a blood product (e.g., serum, plasma, or the like), urine, nasal swabs, a liquid biopsy sample, skin swabs, lesion swabs, or combinations thereof. In some cases, DNA may be extracted from lesion material, such as lesion fluid on a dray swab, lesion fluid swab in viral transport media, lesion fluid on a slide, crust or lesion roof. The term “blood” encompasses whole blood, blood product or any fraction of blood, such as serum, plasma, buffy coat, or the like as conventionally defined. Suitable samples include those which are capable of being deposited onto a substrate for collection and drying including, but not limited to: blood, plasma, serum, urine, saliva, tear, cerebrospinal fluid, organ, hair, muscle, puss, or other tissue samples or other liquid aspirates. The term “biological sample” further refers to a sample obtained from a biological source, including, but not limited to, an animal, a cell culture, an organ culture, tissue, and the like.

As used herein, the terms “substantially,” “approximately,” and “about” are defined as being largely but not necessarily wholly what is specified (and include wholly what is specified) as understood by one of ordinary skill in the art. In any disclosed embodiment, the term “substantially,” “approximately,” or “about” may be substituted with “within [a percentage] of” what is specified, where the percentage includes 0.1, 1, 5, 10, 15, and 20 percent.

As used herein, the terms “swab” and “dry swab” refer to a physical vector for containing and/or obtaining a sample from an individual. The vector may be constructed of various materials and may encompass cotton swab balls, tissues, plastic forceps, plastic inoculating loops, popsicle sticks, dry or moist swab sticks, and others of the like.

### III. Virus Detection and Linear Assignment Techniques

One or more embodiments described herein can be implemented using operative, actionable, or programmatic systems, subsystems, modules, blocks, or components. An operative or actionable system, subsystem, module, block, or component can include in vitro or in silico operations or actions that can be performed by an operator (e.g., a researcher or a practitioner), a machine (e.g., a computing device or a sequencer), or a combination thereof. A programmatic system, subsystem, module, block, or component can include a program, a sub-routine, a portion of a program, or a software component or a hardware component capable of performing one or more stated tasks or functions. As used herein, a system, subsystem, module, block, or component can exist on a hardware component independently of other systems, subsystems, modules, blocks, or components. Alternatively, a system, subsystem, module, block, or component can be a shared element or process of other systems, subsystems, modules, blocks, or components.

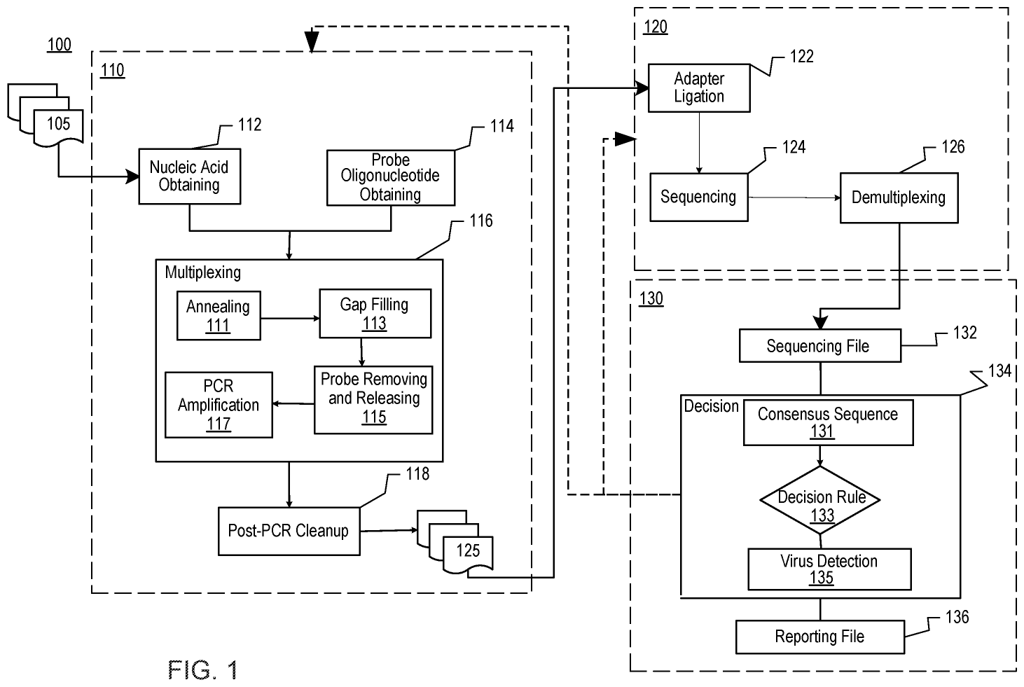

FIG. 1 is a block diagram of a system 100 that is capable of performing the virus detection and lineage assignment techniques described herein. The system 100 is a dynamic clinical assay pipeline that comprises a molecule capture subsystem 110, a sequencing subsystem 120, and an analysis subsystem 130. The system and subsystems, modules, blocks, or components (e.g., program, code, or instructions) illustrated in FIG. 1 may be executable by an operator (e.g., a human user), a specialized machine (e.g., a sequencer), or one or more processors (e.g., a CPU, GPU, or the like) with assistance or use of a computing device according to various embodiments. In some instances, the virus to be detected is a Coronavirus, an Influenza virus, or a Respiratory Syncytial Virus (RSV). In some instances, the virus to be detected is a non-variola Orthopoxvirus, for example, a Monkeypox or Cowpox virus. In other instances, the virus to be detected is a variola Orthopoxvirus, for example, a smallpox virus. The virus is usually thousands to millions bases or base pairs in length. For example, the genome of Monkeypox is about 190,000 base pairs, and the genome size of SARS-CoV-2 is approximately 30,000 bases (30,000 nucleotides).

The molecule capture subsystem 110 is a wet-lab subsystem where chemicals, drugs, or other material or biological matter are tested and analyzed requiring water, direct ventilation, and specialized piped utilities. One or more blocks, modules, or services may be comprised by the molecule capture subsystem 110. For example, as illustrated in FIG. 1, the molecule capture subsystem 110 includes a nucleic acid obtaining block 112, a probe oligonucleotide obtaining block 114, a multiplexing module 116 that comprises an annealing block 111, a gap filling block 113, a probe removing and releasing block 115, a PCR amplification block 117, and a post-PCR cleanup block 118. The input to the molecule capture subsystem 110 are one or more samples 105 and the output of the molecule capture subsystem 110 are a plurality of amplified molecules 125.

At the nucleic acid obtaining block 112, nucleic acid from the one or more samples 105 are obtained. The one or more samples 105 may be collected or obtained from a subject before obtained at the nucleic acid obtaining block 112. Appropriate sample collection methods may be used depending on the sample type and the downstream applications. For example, venipuncture or fingerstick methods can be used for collecting for blood samples, and saliva samples may be collected using available collection kits. In some embodiments, the one or more samples 105 are collected using a kit for self-collection of samples, e.g., the Monkeypox PCR Test Home Collection Kit. The Monkeypox PCR Test Home Collection Kit is intended for use by individuals presenting with acute, generalized pustular or vesicular rash suspected of Monkeypox illness for self-collection of lesion swab specimens in media at home. The swab specimen is placed in media and transported to the laboratory for testing non-variola Orthopoxvirus DNA extracted from the specimens.

The nucleic acid from the one or more samples 105 may be obtained by isolation and extraction. For example, proper laboratory sample preparation methods may be used to break down cells and tissues to release the nucleic acid and remove contaminants such as proteins, lipids, and other cellular debris. Various sample preparation methods are available, such as phenol-chloroform extraction, column-based purification, or magnetic bead-based purification. Once the sample is prepared, the nucleic acid needs to be extracted using appropriate extraction methods. The choice of extraction method may depend on the type of nucleic acid of interest (e.g., DNA or RNA) and the downstream applications. Commonly used extraction methods include organic extraction, silica-based column purification, or magnetic bead-based extraction. Proper quality control methods may also be performed at the nucleic acid obtaining block 112 to check the quality of the obtained nucleic acid. In some instances, the one or more samples 105 may be nucleic acid samples and no preparation or extraction methods are needed. For example, the one or more samples 105 may be nucleic acid obtained from a patient.

At the probe oligonucleotide obtaining block 114, one or more probe oligonucleotides are prepared and obtained. The probe oligonucleotides may be pre-designed to be suitable for capturing target molecules to detect a presence or absence of a virus in a sample. For example, if the virus to be detected is a Monkeypox virus, the probe oligonucleotides may be designed to be suitable for capturing a DNA molecule of the Monkeypox virus that is a predetermined number of base pairs in length (e.g., about 675 base pairs in length). The pre-designed probe oligonucleotides may then be synthesized using appropriate methods such as solid-phase synthesis, enzymatic synthesis, or chemical synthesis. The synthesized probe oligonucleotides are obtained at the probe oligonucleotide obtaining block 114. The probe oligonucleotide obtaining block 114 may only perform the function of obtaining probe oligonucleotides. The design and the synthesis/manufacture of the probe oligonucleotides may be performed by a separate system, subsystem, or block.

At the multiplexing module 116, functions including annealing, gap filling, probe removing and releasing, and amplifying may be performed at different blocks. For example, the annealing block 111 may anneal the probe oligonucleotides obtained at block 114 to target molecules. Annealing is a critical step in hybridization assays that allows the probe oligonucleotides to bind specifically to its target sequences. Different probe oligonucleotides may be designed and annealed to different target molecules at the annealing block 111. When the target molecule is a double-stranded DNA molecule, the corresponding probe oligonucleotide to capture the DNA molecule may be a double-stranded oligonucleotide. If the target molecule is a single-stranded RNA molecule, the corresponding probe oligonucleotide may be a single stranded DNA molecule and annealed to the RNA molecule to obtain an RNA-DNA hybrid duplex that the RNA strand and the DNA strand are complementary to each other. The annealing process may result in a formation of a duplex structure for subsequent steps or processes.

At the gap filling block 113, a gap or a missing section of a DNA or an RNA strand may be filled. Appropriate methods may be used to perform gap filling including synthesis by polymerase and homologous recombination.

At the probe removing and releasing block 115, probes that are not reacted with any target molecules are removed and remaining probes are released from the hybrid molecules. The removal of non-reacted probes may be performed using a variety of methods including washing the mixture with a buffer solution that disrupts the probe-target interaction or using enzymatic digestion to degrade the probe or target sequence. Probe releasing may be conducted by heating, denaturing, or enzyme digestion.

At the PCR amplification block 117, target molecules may be amplified using PCR techniques. Primers that are complementary to flanking regions of target molecules, polymerase enzymes, and deoxynucleotide triphosphates (dNTPs) may be used to perform the PCR amplification. The primers may be served as starting points for polymerase to synthesize a new DNA or RNA strand during the PCR amplification, and different polymerase enzymes may be used to bind to the primers to synthesize the new DNA or RNA strand.

At the post-PCR cleanup block 118, unwanted components are removed. It is often necessary to perform a post-PCR clean-up step to remove unwanted reaction components, such as primers, nucleotides, enzymes, and other impurities. Different post-PCR cleanup methods including gel electrophoresis, bead-based purification, and spin column purification may be used at the post-PCR cleanup block. The post-PCR clean-up can improve the accuracy and efficiency of downstream applications. After the post-PCR clean-up step, the amplified molecules 125 are obtained and may be used for subsequent sequencing and analysis subsystems. Target molecules are amplified thousands to millions fold. In some instances, the number of the amplified molecules is about 16 million times of the number of the target molecules.

The sequencing subsystem 120 may be performed in a sequencer, which is an automated system capable of sequencing DNA or RNA molecules and analyzing genetic fragments for a variety of applications. Examples of the sequences that may be used to execute the sequencing subsystem 120 may include Sanger sequencers, Illumina sequencers, PacBio sequencers, Oxford Nanopore sequencers, Ion Torrent sequencers, and the likes. For example, the sequencing subsystem 120 may be a capillary electrophoresis-based system, where genetic fragments bound to probes migrate through a polymer and the fluorescence emissions are measured. An array of multiple capillaries allows for sample loading in a multi-well microplate format. The sequencing subsystem 120 may also be a pyrosequencing technology-based system for rapid sequencing and analysis. Pyrosequencing is a method of DNA sequencing (in some instances, RNA sequencing) based on the “sequencing by synthesis” principle, in which the sequencing is performed by detecting the nucleotide incorporated by a DNA polymerase. Pyrosequencing relies on light detection based on a chain reaction when pyrophosphate is released. The modules, blocks, or components may be stored on a non-transitory computer medium. As needed, one or more of the modules, blocks, or components may be loaded into system memory (e.g., RAM) and executed by one or more processors the sequencing subsystem 120. One or more blocks, modules, or services may be comprised by the sequencing subsystem 120. For example, as illustrated in FIG. 1, the sequencing subsystem 120 includes an adapter ligation block 122, a sequencing block 124, and a demultiplexing block 126.

At the adapter ligation block 122, an adapter may be ligated to a molecule of the amplified molecules 125. During the adapter ligation, the adapters are ligated to the ends of the molecules using ligase (e.g., DNA ligase). For example, the adapters that are used at the adapter ligation block 122 may be SMRTbell adapters. SMRTbell adapters are adapter sequences used in PacBio's single-molecule real-time (SMRT) sequencing technology. The SMRTbell adapter consists of two complementary oligonucleotide strands that can be annealed to the molecule ends and then ligated together to form a circular molecule. The circular molecule can then be sequenced using PacBio's SMRT sequencing technology. Other suitable adapters may also be used at the adapter ligation block 122.

At the sequencing block 124, the molecules (e.g., circular molecules) obtained from the adapter ligation block 122 are sequenced using appropriate sequencing techniques to obtain sequence reads. This process usually obtains thousands to millions or millions to billions of sequence reads. Circular consensus sequencing (CCS) techniques that enable the DNA polymerase to repeatedly pass over the same region of the circular molecule can be used at the sequencing block 124. CCS sequencing used at the sequencing block 124 also ensures longer read lengths and higher consensus accuracy. In some instances, a consensus sequence read is generated based on raw sequence reads. The consensus sequence reads may be referred as “sequence reads” herein. It should be understandable that other sequencing techniques such as nanopore sequencing may also be used at the sequencing block 124. to provide sequence reads that are suitable for applying the disclosed techniques.

At the demultiplexing block 126, the sequence reads obtained at the sequencing block 124 may be separated by samples (or molecules). Demultiplexing is a process of separating pooled sequencing reads based on sample-specific barcodes or indices that were added to each sample during library preparation. When the sequencing involves multiple samples, the sequencing reads from different samples are often pooled together and sequenced in a single run at the sequencing block 124. To separate the sequence reads from each sample, demultiplexing is performed. In some instances, the demultiplexing also comprise an adapter trimming step. In other instances, adapters have been trimmed at the sequencing block 124.

The analysis subsystem 130 may be executed in a computing device such as a computer, a central processing unit (CPU), a graphics processing unit (GPU), or the like. The modules, blocks, or components of the analysis subsystem 130 may be stored on a non-transitory computer medium. As needed, one or more of the modules, blocks, or components may be loaded into system memory (e.g., RAM) and executed by one or more processors in the analysis subsystem 130. One or more blocks, modules, or services may be comprised by the analysis subsystem 130. For example, as illustrated in FIG. 1, the analysis subsystem 130 includes a sequencing file block 132, a decision module 134, and a reporting file block 136.

At the sequencing file block 132, a sequencing file is generated by the computing device where the analysis subsystem 130 is executed. The sequencing file may be a FASTQ file, a BAM file, a SAM file, a FASTA file, a CRAM file, or the like. The generation of the sequencing file is based on the output of the sequencing subsystem 120. The sequencing file generally contains sequencing information, for example, sequence reads of each amplified molecule and the positioning data associated with the sequence reads. The positioning data may comprise a start/end position of each sequence read in a reference virus genome. In some instances, the positioning data are determined by mapping or aligning each sequence read to the reference virus genome. Different alignment algorithms may be used for the alignment. The sequencing file may be output to the decision module 134 for virus detection and/or lineage assignment.

At the decision module 134, functions comprising generating consensus sequences, determining or obtaining decision rules, and determining a presence or absence of the virus and/or a lineage of the virus may be performed at different blocks. For example, as shown in FIG. 1, the decision module 134 comprises a consensus sequence block 131, a decision rule block 133, and a virus detection block 135.

At the consensus sequence block 131, a consensus sequence for each target molecule is generated. Different consensus sequence generating rules may be used to generate the consensus sequence at the consensus sequence block 131. In some instances, a nucleotide identity is assigned to a position in the consensus sequence if at least a predetermined number of the sequence reads has the nucleotide identity in the position, and an “N” is assigned to a position in the consensus sequence if less than a predetermined number of the sequence reads has the nucleotide identity in the position. In some instances, the predetermined number is four. The wet-lab and sequencing subsystems guarantee the read coverage so that a generating method based on consistent reads works efficient and effective to decide the consensus sequence. In most cases, at least 4 read coverage is guaranteed in 80% of the positions in the reference virus genome.

At the decision rule block 133, decisions rules regarding the virus detection and/or lineage assignment are obtained. The decision rules may be predetermined by researchers, doctors, practitioners, or any authorized persons. The decision rules may also be determined using the computing device based on an algorithm or a machine learning model. In some instances, a consensus sequence is mapped to the reference genome or a portion of the reference genome, and one or more scores are decided based on the decision rules. In some instances, the one or more scores are decided based on a distribution of nucleotide comparisons (e.g., nucleotide substitutions, nucleotide deletions, nucleotide insertions and the like) between the consensus sequence and the reference genome or the portion thereof. For example, the distribution shows count of nucleotide substitutions, nucleotide deletions, nucleotide insertions and the like. The resolution of the nucleotide comparisons may differ. In some instances, the nucleotide comparisons are single-nucleotide comparisons. In other instances, the nucleotide comparisons are multiple-nucleotide comparisons (e.g., three-nucleotide comparisons). Different scores may be assigned to different types of nucleotide comparisons. In some instances, different scores are assigned to subtypes in the same type of nucleotide comparisons. For example, the assigned score for a substitution of “A” to “G” may be different from the assigned score for a substitution of “A” to “C.” In some instances, different scores are assigned based on the phylogenetic context (e.g., the evolutionary relationships and lineage classifications) of the nucleotide comparisons. For example, nucleotide substitutions that are common in specific lineages may receive different scores than those that are rare. In some instances, nucleotide comparisons that lead to changes in the encoded amino acids in protein-coding regions may receive higher scores, especially if the change is known to affect the function of the protein. In some instance, a same score is assigned to different types of nucleotide comparisons.

The decision rules may comprise a similarity metric that illustrates the relationship between consensus sequences and the reference genome (or a reference sequence). In some instances, the similarity metric assigns different weights to the one or more scores associated with different consensus sequences. For example, the weight assigned to exact matches between a consensus sequence and the reference genome is different from the weight assigned to mismatches between the consensus sequence and the reference genome. In some instances, the similarity metric assigns positive weights to a total number of mutations in the consensus sequences and to a total number of mutations in the reference genome, and assigns negative weights to nucleotide comparisons between the consensus sequences and the reference genome. The decision rules may also comprise a threshold for determining a presence or absence of the virus and/or a threshold for determining the predicted lineage assignment. In some instances, if the one or more score is greater than or equal to the threshold, the virus is determined to be detected or the predicted lineage is determined to be assigned to the virus in the sample. If the one or more score is less than the threshold, the virus may be determined to be absent in the sample or the lineage is determined to be not assigned to the virus in the sample.

The decision rules may comprise a decision tree model. In some instances, the decision tree model is an annotated phylogenetic tree that comprises different lineages as nodes. In some instances, the decision rules are stored in a tree-like data structure, e.g., a binary tree, a binary search tree, a trie, a n-ary tree, a general tree, a B-tree, an XML, tree, or the like. Storing decision rules in a tree-like data structure can improve efficiency in data searching (e.g., decision making) and can be space-efficient. Tree structures can easily adapt to dynamic data, allowing for efficient updates, insertions, and deletions. This makes them versatile for data that changes over time, and fits the dynamic clinical assay pipeline design as disclosed herein. The tree structures also provide the feasibility of visualizing the virus lineage assignments.

At the virus detection block 135, a presence or absence of the virus in the sample and/or a predicted lineage of the virus is determined. In some instances, both the presence or absence of the virus in the sample and the predicted lineage of the virus are determined based on the consensus sequences of target molecules generated at the consensus sequence block 131 and the decision rules that are obtained at the decision rule block 133. In some instances, the presence or absence of the virus in the sample is determined or pre-determined by wet-lab means, e.g., using real-time PCR, viral culture, immunofluorescence assay, electron microscopy, or the like. If the virus has been pre-determined to be present in the sample (e.g., using real-time PCR or based on a decision rule), a predicted lineage assignment of the virus in the sample is determined at the virus detection block 135. The predicted lineage assignment of the virus in the sample may be determined by assigning a lineage to the virus in the sample based on a decision tree model, e.g., the decision tree model obtained at block 133.

In some instances, the predicted lineage is assigned based on the similarity metric. One or more scores of the consensus sequences may be determined at the virus detection block 135 based on the decision rules obtained at block 133 and the one or more scores are used for determining the presence or absence of the virus in the sample and/or the predicted lineage assignment of the virus in the sample. In some instances, the one or more scores are compared against each other, and a known lineage associated with the highest or lowest score will be assigned to the virus in the sample. In some instances, when each of the one or more scores exceeds a predetermined threshold, an unknown lineage will be assigned to the virus in the sample (or marked as “new” lineage), and further validation will be performed to confirm the existence of this unknown lineage. The validation may be performed by peer review, reproducing using sequencing techniques, data analysis, and phylogenetic methods (e.g., the pipelines as disclosed herein), or the like. In some instances, bioinformatics tools such as NextClade and Pangolin may be used or modified to determine the one or more scores, or the presence or absence of the virus in the sample and/or its predicted lineage assignment.

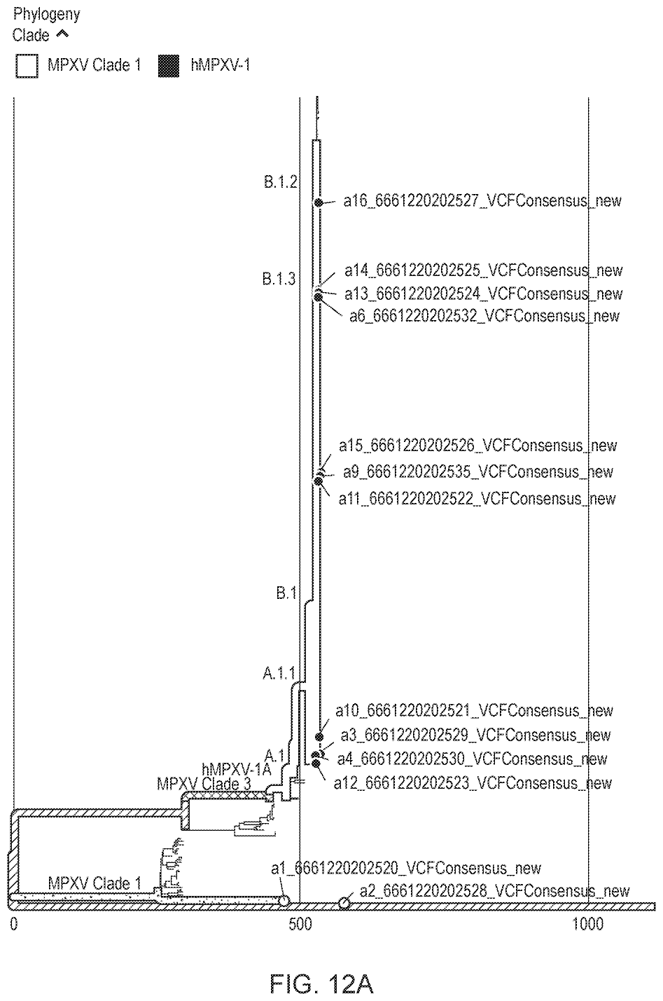

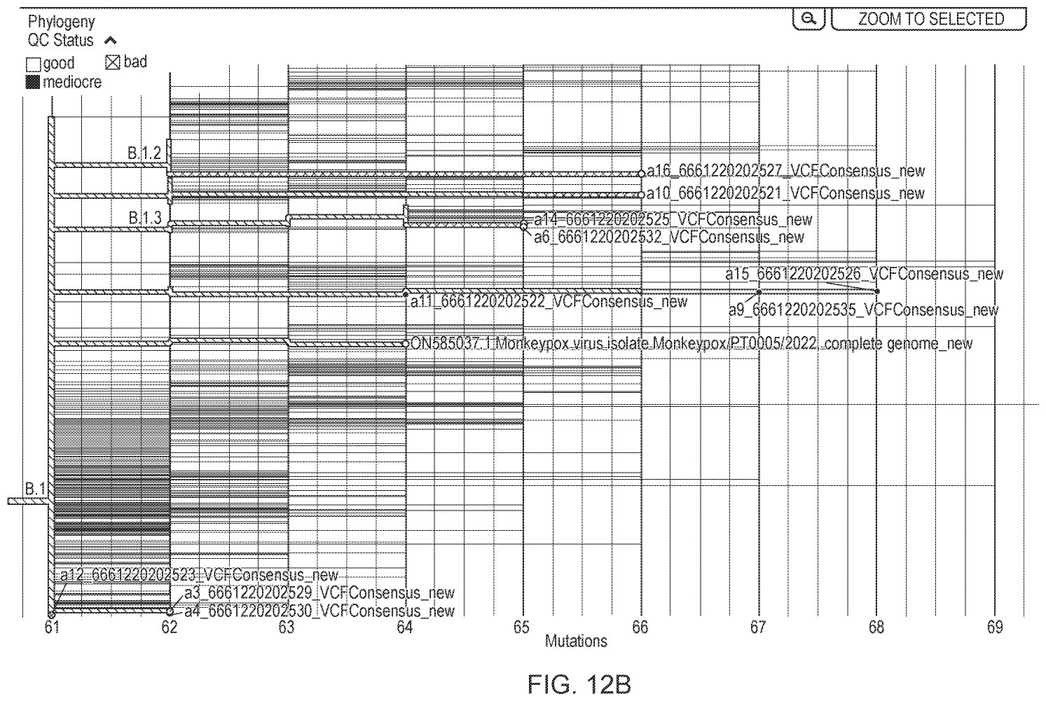

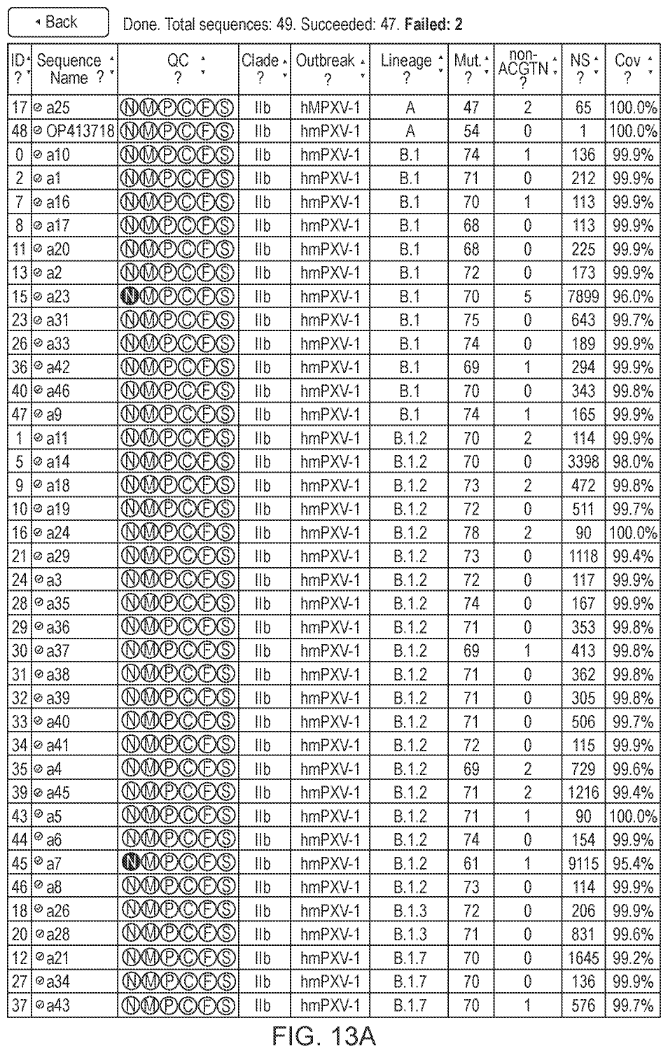

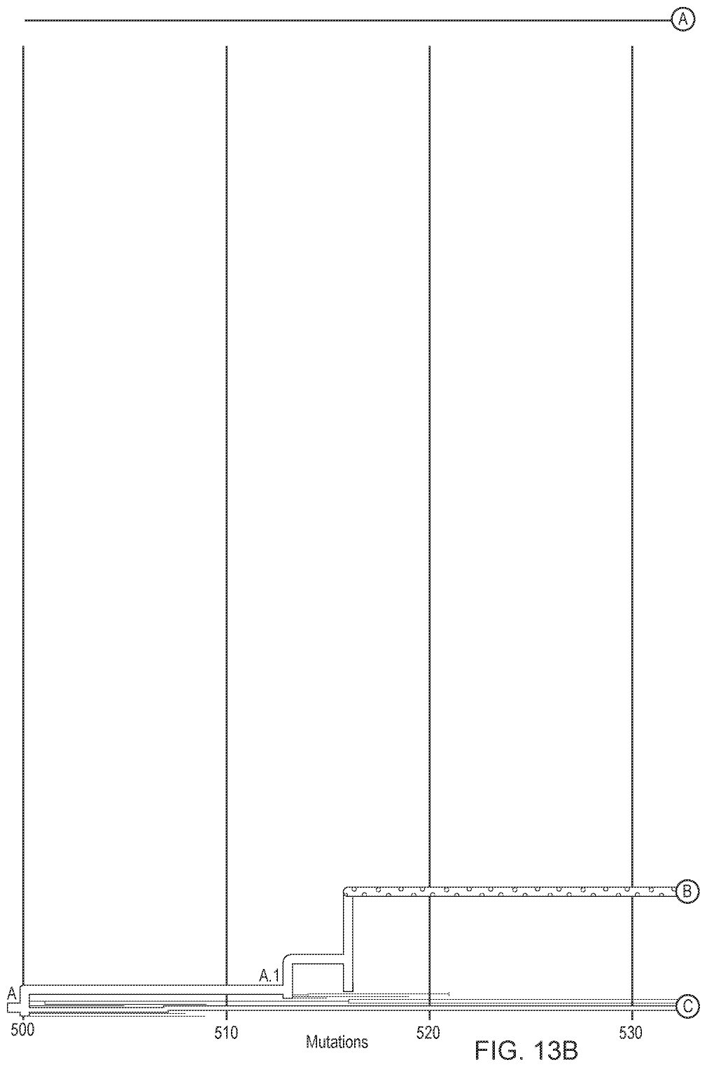

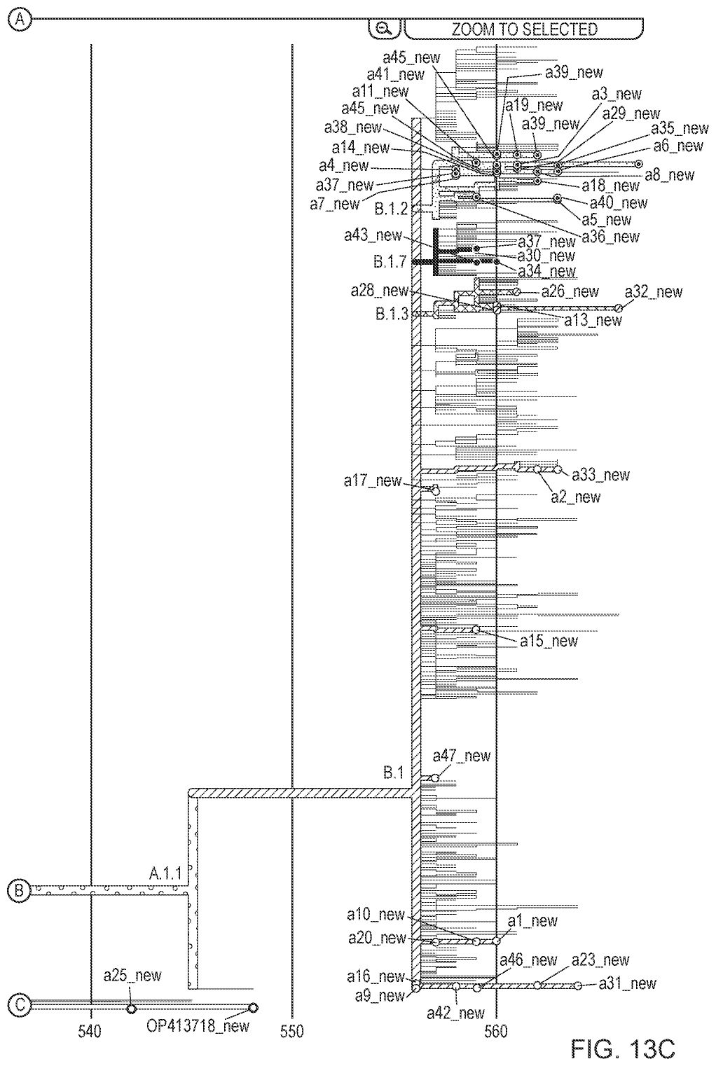

At the reporting file block 136, a reporting file is generated by the computing device where the analysis subsystem 130 is executed. The reporting file may be a VCF (Variant Call Format) file, a VCFCons file, a BCF file, a MAF file, a GFF file, or the like. The generation of the reporting file is based on the output of the decision module 134. In some instances, the reporting file comprises (i) a presence or absence of the virus in the sample, and/or (ii) a predicted lineage of the virus in the sample. In some instances, the reporting file is output to a graphic user interface (GUI). In some instances, the reporting file also comprises a display of lineage assignment based on the decision rules obtained at block 133 (e.g., see FIGS. 12A-12B, and 13B-13C).

In some instances, the information generated in the decision module 134 is used to dynamically modify the molecule capture subsystem 110 and/or the sequencing subsystem 120. For example, based on the lineage assignment determined during a period of time, a prevalence of a lineage can be determined. Based on the prevalent lineage, the probes used in the molecule capture subsystem 110 may be reselected or redesigned to better capture target molecules corresponding to the prevalent lineage. Adapters used in the sequencing subsystem 120 may also be reselected or redesigned to adapt to the change in probes/target molecules. The dynamic modification of the system 100 via feedback from the decision module 134 to the molecule capture subsystem 110 and/or the sequencing subsystem 120 helps reconstruct the dynamic clinical assay pipeline in real time. The dynamic modification of the system 100 also improves capture and sequencing efficiency for using a known or estimated prevalence of a lineage. The dynamic modification of the system 100 also contributes to provide accurate virus detection and lineage assignment results.

For example, if Lineage B.1 is found to be prevalent for a subject population (e.g., defined by a particular race, a particular age range, a particular geographical location, or the like), the one or more probe oligonucleotides that are prepared and obtained at block 114 can be re-designed to be suitable for capturing target molecules that are specific to Lineage B.1. The adaptors used at the adapter ligation block 122 may also be updated based on the target molecules that are specific to Lineage B.1. Additionally, the decision rules obtained at block 133 can be accordingly updated to reflect the prevalence of Lineage B.1. These updates enhance the precision of predictive outcomes. Furthermore, this optimization serves to significantly increase the efficiency and cost-effectiveness of future predictions and assignments regarding virus lineages for samples collected from the subject population. These dynamic adjustments and enhancements represent a substantial improvement in the field of virus detection and lineage assignment, as well as in the domain of virus assay procedures.

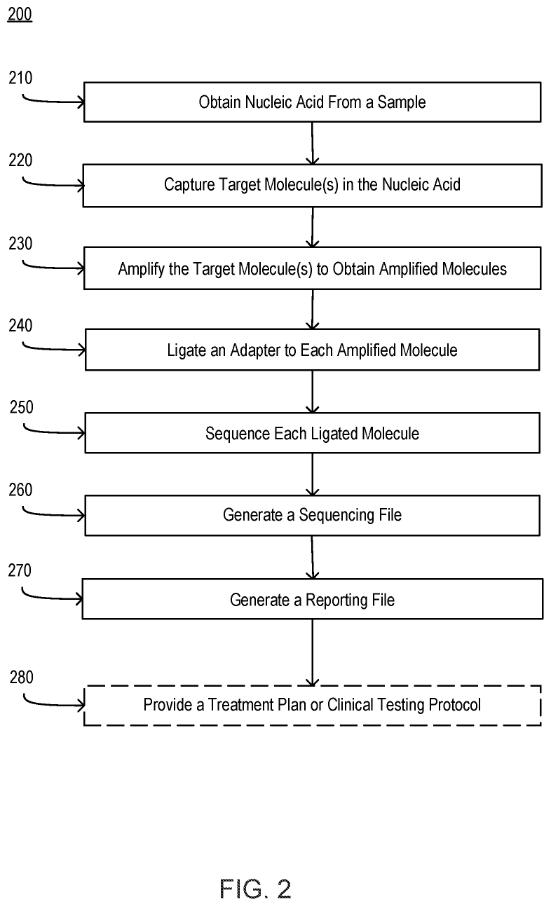

FIG. 2 is an exemplary flowchart showing a process 200 of generating a reporting file concerning a presence or absence of a virus or a predicted lineage of a virus in a sample in accordance with various embodiments. In certain embodiments, the processing or a portion of the processing depicted in FIG. 2 may be performed by system 100 as described with respect to FIG. 1. Process 200 begins at block 210, where nucleic acid is obtained from a sample. The sample may be pre-obtained from a subject or a living organism such as a plant, an animal, or a human being. The sample may be a blood sample, a urine sample, a tissue sample, a saliva sample, a feces sample, a hair sample, a bone sample, or the like. The nucleic acid obtained may comprise deoxyribonucleic acid (DNA) and/or ribonucleic acid (RNA). A variety of nucleic acid obtaining methods may be used at block 210 to obtain the nucleic acid from the sample.

At block 220, target molecules in the nucleic acid are captured. In some embodiments, the target molecules are DNA molecules. In some embodiments, the target molecules are RNA molecules. In some embodiments, the target molecules are a mixture of DNA molecules and RNA molecules. In various embodiments, multiple target molecules in the nucleic acid are captured at block 220. In other embodiments, one target molecule in the nucleic acid may be captured. A variety of molecule capture methods may be used to capture the target molecules. In various embodiments, the target molecules are captured using probe hybridizations. In other embodiments, the target molecules are captured using methods such as affinity capture, chromatography, microfluidics, and the like.

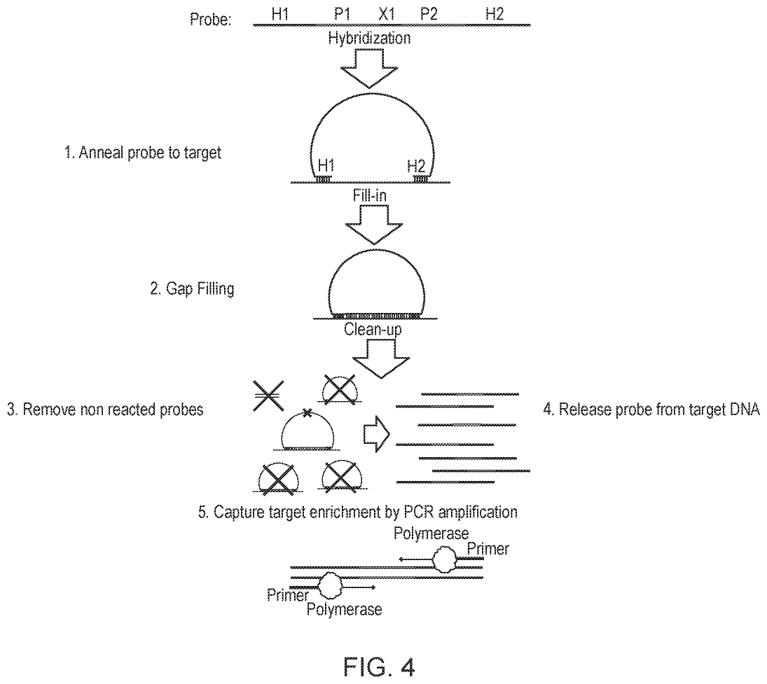

At block 230, the captured molecules are amplified to obtain amplified molecules. In various embodiments, the captured molecules are amplified using polymerase chain reaction (PCR) techniques. Other methods such as isothermal amplification, multiple displacement amplification (MDA), rolling circle amplification (RCA), digital PCR (dPCR), reverse transcription polymerase chain reaction (RT-PCR), digital droplet PCR (ddPCR), or the like may also be used at block 230 to amplify the captured molecules. Substantially unique barcodes may be attached to the captured molecules before amplification so that information can be traced back. In some embodiments, a same barcode is added to target molecules in the same sample. In some embodiments, each target molecule has its own barcode and the barcodes can be used to distinguish target molecules. The barcode sequences may be predetermined to reduce lab costs and improve lab preparation efficiency. The barcode sequences may also be randomly generated to guarantee the substantial uniqueness. The steps at block 220 and block 230 may be performed in a Molecular Inversion Probe Set Design and PCR Pipeline, which is disclosed in detail in Section IV and FIG. 4.

At block 240, the amplified molecules are ligated to adapters for efficient sequencing. In some embodiments, the adapters are SMRTbell adapters with selected sizes. The SMRTbell adapters are adapters used in single-molecule, real-time (SMRT) sequencing technology. They have a hairpin structure, with the 5′ and 3′ ends of the adapters coming together to form a loop. This structure allows the SMRTbell adapters to circularize, which is important for SMRT sequencing as it allows for multiple passes of the polymerase over the same molecule. Each SMRTbell adapter may also comprise a substantially unique barcode/sequence that is used for sample identification and demultiplexing during data analysis. This allows multiple samples to be sequenced together in a single SMRT cell, which improves the sequencing technology by increasing sequencing efficiency and decreasing per sample cost. Primer annealing and polymerase binding steps may also be performed at block 240. Ligated molecules are generated at block 240. In some embodiments, the ligated molecules are circular molecules.

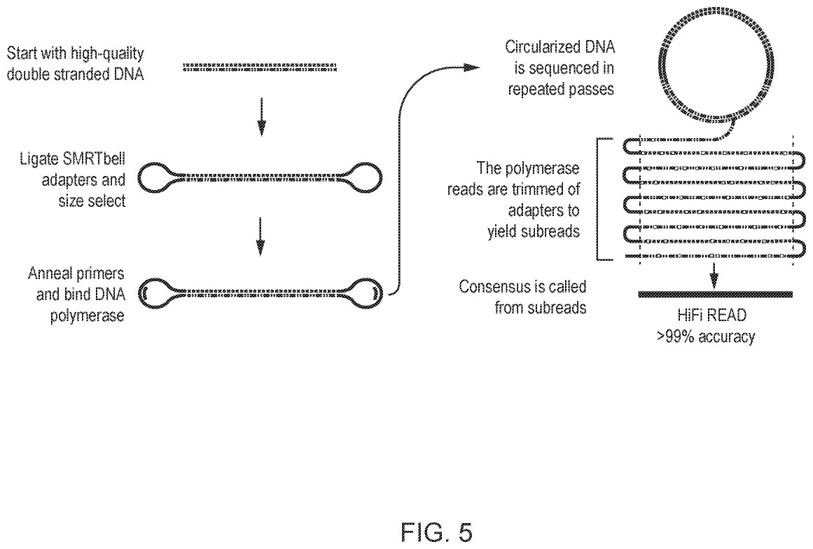

At block 250, the ligated molecules are sequenced using appropriate sequencing methods to obtain sequence reads. In some embodiments, the sequencing method is a circular sequencing. In some embodiments, the sequencing is a SMRT sequencing. Other sequencing methods such as RCA sequencing or nanocircle sequencing may also be used for sequencing the ligated molecules. The steps at block 240 and block 250 may be performed in an Adapter Design, Ligation, and Sequencing Pipeline, which is disclosed in detail in Section V and FIG. 5.

At block 260, a sequencing file is generated. The sequencing file may comprise information pertaining to the sequencing step at block 250. In some embodiments, the sequencing file comprises the sequence reads of each ligated molecules and a position of each sequence read in a reference genome of a virus by aligning the sequence read to the reference genome of the virus. In some embodiments, the sequencing file is a BAM file. The sequencing file may be generated by a computing device or a sequencer.

At block 270, a reporting file is generated. The reporting file may be generated by the same computing device as the one generates the sequencing file. The reporting file comprises information pertaining to a presence or absence of the virus in the sample and/or its predicted lineage. A consensus sequence for each target molecule may be determined at block 270. Determining the consensus sequences can increase the accuracy in determining the presence or absence of the virus in the sample and/or its predicted lineage. In some embodiments, a genome construct may be determined based on the consensus sequences of target molecules and the genome construct may be used to determine the presence or absence of the virus in the sample and/or its predicted lineage. In some embodiments, the sample is known to contain viral nucleic acid and only the lineage of the virus in the sample is determined. One or more scores may also be determined at block 270 based on decision rules that are predetermined or determined based on an algorithm or a machine learning model. The presence or absence of the virus in the sample and/or its predicted lineage may be determined at block 270 based on the one or more scores. In some embodiments, the reporting file is a VCF file or a modified VCF file. In some embodiments, the reporting file may be output to a GUI to a practitioner or the subject. The steps at block 260 and block 270 may be performed in an Adjusted VirSeq Pipeline, which is disclosed in detail in Section VI.

At optional block 280, a treatment plan or clinical testing protocol is provided. The treatment plan or clinical testing protocol may be determined based on the one or more scores, the presence or absence of the virus in the sample and/or its predicted lineage, or the reporting file generated at block 270. In some embodiments, the virus is a Monkeypox (MPX) virus and the treatment plan comprises administering antiviral medications for the subject.

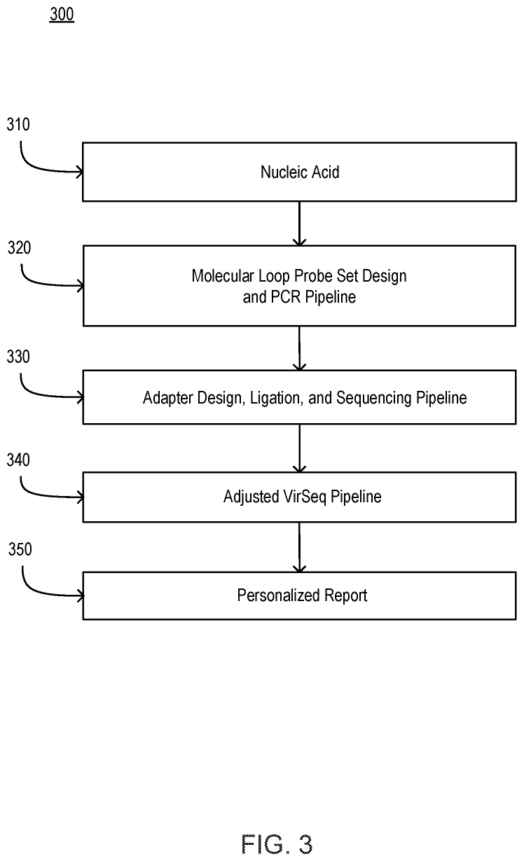

FIG. 3 is an exemplary flowchart showing a process 300 of performing disclosed techniques concerning a presence or absence of a virus or a predicted lineage of a virus in a sample in accordance with various embodiments. In certain embodiments, the processing or a portion of the processing depicted in FIG. 3 may be performed by system 100 as described with respect to FIG. 1. The process 300 can be conducted in three pipelines. Nucleic acid is obtained at block 310 and sent to the Molecular Loop Probe Set Design and PCR Pipeline at block 320 to obtain amplified molecules. The amplified molecules are then sent to the Adapter Design, Ligation, and Sequencing Pipeline at block 330 to obtain sequence reads, and the sequence reads are sent to the Adjusted VirSeq Pipeline to obtain a personalized report. The personalized report is output at block 350. Details to the process 300 is described in detail in Sections IV, V, and VI.

### IV. Molecular Inversion Probe Set Design and PCR Pipeline

The Molecular Loop Probe Set Design and PCR Pipeline may correspond to the subsystem 110 in FIG. 1 and is further illustrated in FIG. 4. The Molecular Loop Probe Set Design and PCR Pipeline is designed to capture target molecules and amplify the captured molecules so that a sufficient number of molecules can be sent to the Adapter Design, Ligation, and Sequencing Pipeline to secure a sufficient number of sequence reads and a sufficient coverage regarding the sequence reads and a reference genome. In some embodiments, the sufficient number of molecules/sequence reads is about 4 million to 8 million, and the sufficient coverage is (i) about or greater than 50% coverage (e.g., 50%, 60%, 70%, 80%, 90%), or (ii) about or greater than 100,000 sequence reads (e.g., 100,000 sequence reads, 1 million sequence reads, 10 million sequence reads).

In order to detect a presence or absence of a virus or a predicted lineage of a virus in a sample, nucleic acid need to be obtained from the sample. The nucleic acid often comprises both the host genetic molecules and the potential or suspicious virus molecules. Therefore, the Molecular Loop Probe Set Design and PCR Pipeline should be able to capture virus molecules in the nucleic acid. In some embodiments, a target molecule to be captured by the Molecular Loop Probe Set Design and PCR Pipeline is a virus molecule. The virus molecule may be a DNA molecule or an RNA molecule. The DNA molecule is a double-stranded molecule while the RNA molecule is often single-stranded. In some embodiments, the virus to be detected is a MPX virus and the MPX virus is a double-stranded DNA virus.

To capture a target molecule, a probe hybridization method may be used. The Molecular Loop Probe Set Design and PCR Pipeline may comprise a component to design probes to capture the target molecule. Appropriate design methods may be used to capture target molecules, and the methods may depend on various factors including the type of molecules, the sensitivity and specificity requirements, and the downstream analysis needs. For example, when the virus to be detected is a MPX virus and the target molecule is a MPX molecule, a probe may be designed to consist of two binding sites about 600-700 base pairs (bp) apart. In some embodiments, a probe to detect a MPX molecule consists of two binding sites that are 675 bp apart. In some embodiments, a plurality of probes is designed to capture a plurality of target molecules. For example, to capture MPX virus molecules, more than 7,000 probes (e.g., 7,826) may be designed to capture 675 bp molecules that are evenly tiled in the MPX genome that is about 200 kilobase pairs (kb).

In some embodiment, probes to capture target molecules are molecular inversion probes. Molecular inversion probes may provide a higher specificity and sensitivity in capturing target molecules compared to some other types of probes. For example, molecular inversion probes are designed to hybridize to a specific molecule or a genomic region of interest, and the specificity reduces chances of false positives or false negatives in downstream analysis. Molecular inversion probes are also able to detect low levels of DNA or RNA molecules in nucleic acid due to their high signal-to-noise ratio. Additionally, molecular inversion probes can be designed to capture multiple target molecules or genomic regions simultaneously, which allows for high-throughput analysis of multiple targets. Moreover, molecular inversion probes are cost-effective compared to some other target-capturing methods. Molecular inversion probes are also easy to design and can be customized to fit specific research and industrial/clinical needs.

FIG. 4 illustrates a portion of the Molecular Loop Probe Set Design and PCR Pipeline for capturing target DNA molecules in accordance with various embodiments. As shown in FIG. 4, a molecular inversion probe may be designed to have two target complementary regions H1 and H2, two PCR primers P1 and P2, and a probe-release cleavage site X1. In the probe hybridization process, as shown in Step 1 of FIG. 4, a plurality of probes may be added to the nucleic acid obtained from the sample, where each probe is annealed to a target molecule on H1 and H2 regions. The molecular inversion probes may be tiled across greater than 99% (e.g., 99.6%) of the 200 kb MPX viral genome and consist of two binding sites approximately 675 bp apart.

Molecular inversion probes can be beneficial in capturing target molecules. Firstly, they allow tiling and prevent drop out from novel genome variations. Secondly, unlike other probe-based assays that require sheering a genome to capture molecules, no upstream genome processing is required for using molecular inversion probes, and the pipeline using molecular inversion probes can be processed much more quickly and relatively cheaply at high throughput. Additionally, it ensures expected product size that aids in optimizing sequencer loading and analysis pipelines. It also ensures a full coverage (or sufficient coverage) of the genome as well deep coverage of the genome at most of the base positions.

After binding, the region in-between the two probes are synthesized with DNA polymerase and ligated to form a closed molecule, as shown in Step 2 of FIG. 4. The gap is filled by DNA polymerase using free nucleotides and the ends of the probe are ligated by ligase, resulting in a fully circularized probe.

Since the gap filling step is not performed for non-reacted probes, those probes remain linear. As shown in Step 3 of FIG. 4, the non-binding or incomplete loops remain linear molecules and are removed with exonuclease digestion.

As shown in Step 4 of FIG. 4, circular molecules are released and linearized. In some embodiments, the probe-release cleavage site X1 is cleaved by restriction enzymes such that the probe becomes linearized. In this linearized probe the universal PCR primer sequences are located at the 5′ and 3′ ends and the captured genomic target becomes part of the internal segment of the probe. In other embodiments, the probes may be left as a circularized molecule and no cleavage is needed.

The captured molecules may then be enriched via amplification with a 3′ molecular loop specific M13 universal sequence and 5′ sample specific barcodes, as shown in Step 5 of FIG. 4. If the probe is linearized, traditional PCR amplification may be performed to enrich the captured molecules using the universal primers of the probe. Otherwise, circulating amplification methods such as RCA may be performed for the circular probe.

In some embodiments, the adapters P1 and P2 in FIG. 4 may be used for barcode addition. Samples may then be pooled by equal volume and library prepped for sequencing (e.g., PacBio sequencing). Library preparation may entail DNA damage repair, ligation of sequencing adapters, non-ligated product removal by enzymatic digestion, and bead purification. Libraries may then sequenced, for example, on a PacBio Sequel II with 15-hour movies.

In certain embodiments, the target molecules are RNA molecules. The Molecular Loop Probe Set Design and PCR Pipeline is also capable to capture and amplify RNA molecules. In such embodiments, reverse transcriptase may be used to synthesize cDNA from RNA.

### V. Adapter Design, Ligation, and Sequencing Pipeline

The Adapter Design, Ligation, and Sequencing Pipeline may correspond to the subsystem 120 in FIG. 1 and is further illustrated in FIG. 5. The Adapter Design, Ligation, and Sequencing Pipeline may be designed to provide a circular sequencing pipeline to obtain a stable sequence read coverage per site and across genome. In various embodiments, a SMRT sequencing technology may be used to generate sequence reads. In order to use the SMRT sequencing technology, adapters may be designed and ligated to the target molecules to generate circular molecules. The SMRT sequencing technology may increase sequence read length to an average number that is substantially greater than the traditional sequencing methods. In some instances, the SMRT sequencing technology can produce sequence reads with average length of 50 kb. With circularized DNA molecules, this sequence read length may provide about a greater number of consecutive passes (e.g., 70) of a single target molecule (e.g., in the about 600-700 bp size range). Separate passes can be integrated to a single consensus highly accurate consensus sequence (HiFi) read (e.g., 600-700 bp in length) with 99% accuracy.

The sequencing utilizing Single Molecule, Real-Time (SMRT) long-read sequencing technology requires a circular template (sequencing molecules). The circular template generated from library prep is bound with a polymerase and primer and loaded onto the SMRTCell (sequencing cell). A single molecular product diffuses into one of 8 million zero-mode waveguide (ZMWs) wells where the polymerase is immobilized at the bottom. Phospho-linked nucleotides are then introduced to the ZMWs where the base can then be incorporated by the polymerase. When a given base pair is incorporated, its addition produces a nucleotide specific emission of light that is detected on a per well basis by a camera. This process is repeated for a given amount of time, or movie length, and the nucleotide order on a given well is analyzed and translated to the corresponding nucleotide in the long sequence read output.

FIG. 5 illustrates a portion of the Adapter Design, Ligation, and Sequencing Pipeline for generating circular DNA molecules in accordance with various embodiments. As shown in FIG. 5 (see Schematic Workflow in https://ccs.how/, last visited on Mar. 22, 2023), the Adapter Design, Ligation, and Sequencing Pipeline starts with high-quality double-stranded DNA molecules. Hairpin adapters may be ligated to the linear DNA molecules to produce circular DNA molecules. In some embodiments, the adapters are SMRTbell adapters. The size of the adapters may vary. The circular DNA molecules enables the pipeline to produce long sequence reads that can be used to generate HiFi reads. After the adapter ligation, selected primers and DNA polymerase may be added to sequence the circular DNA molecules and obtain sequence reads.

In some embodiments, as shown in the right part of FIG. 5, the long sequence reads are trimmed off adapters to yield subreads and HiFi reads may be generated based on the subreads. There are advantages associated with the HiFi reads. First, the HiFi reads provide both read length and accuracy that are suitable for various downstream analysis such as variant calls. Moreover, the technique also improves sequencing efficiency: it decreases the sequencing time and cost per sample while increasing the throughput and sequencing efficiency.

### VI. Adjusted VirSeq Pipeline

The Adjusted VirSeq Pipeline may correspond to the analysis subsystem 130 in FIG. 1. The Adjusted VirSeq Pipeline is designed to provide a personalized report concerning the virus information in the sample obtained from a subject. The Adjusted VirSeq Pipeline takes the sequence reads from the Adapter Design, Ligation, and Sequencing Pipeline as input and outputs the personalized report. In some embodiments, the personalized report may be displayed in a GUI.

In various embodiments, a sequencing file may be generated based on sequence reads and a reference virus genome. The sequence reads may be raw sequence reads generated by the Adapter Design, Ligation, and Sequencing Pipeline, or the HiFi reads generated using the raw sequence reads. The reference virus genome may be obtained from a virus database, or modified based on a virus genome obtained from the virus database. In some embodiments, the sequencing file is a BAM file. In some embodiments, the sequencing file is a FASTQ file. Software, scripts, and codes may be used to generate the sequencing file.

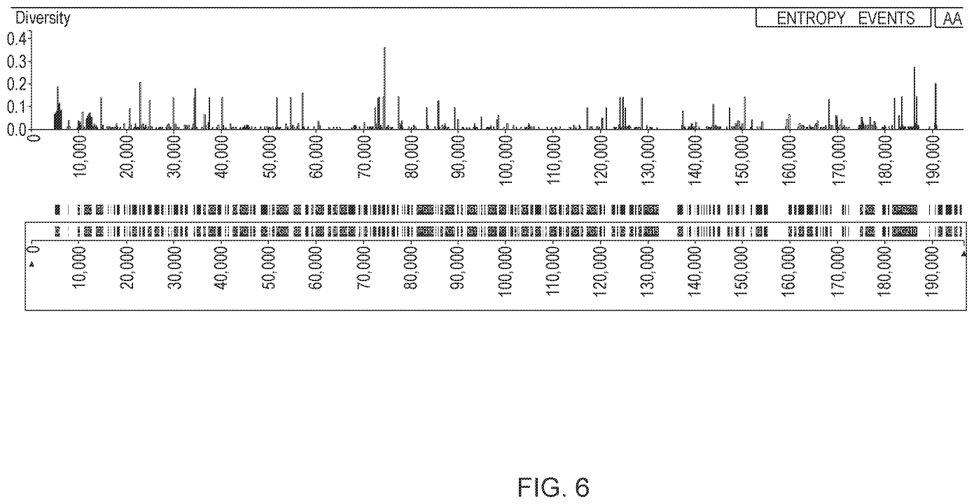

The Adjusted VirSeq Pipeline and the dynamic clinical assay pipeline are designed to be easily adaptable to detect different viruses with a variety range of genome length. In some embodiments, the Adjusted VirSeq Pipeline and the dynamic clinical assay pipeline are designed to detect MPX virus. FIG. 6 shows an overview of the complexities of the MPX genome (see Genomic epidemiology of monkeypox virus, https://nextstrain.org/monkeypox/hmpxy1, last visited on Mar. 22, 2023). The MPX genome is about 10× larger than a SARS-CoV-2 genome and contains lots of long stretches of repetitive DNA and drop regions. More probes in the Molecular Loop Probe Set Design and PCR Pipeline may be designed and put in use to capture target molecules for a virus genome with higher level of complexity. For example, the number of probes needed for a MPX virus detection is about 7×-10× than the number of probes needed for SARS-CoV-2 detection.

In some embodiments, PacBio SMRT LINK software and custom molecular loop processing scripts may be used to generate the sequencing file. The sequencing file may be analyzed using a genome analysis pipeline. In some embodiments, the genome analysis pipeline may be implemented using a CLC genomics server. It should be understood that other sequencing analysis systems may also be used. At this point, the sequencing primer sequences can be removed, and the sequence is aligned to a reference virus genome (e.g., a MPX reference genome) to generate a BAM file of alignment. In certain embodiments, Minimap2 may be used to generate the alignment. Other alignment programs or algorithms may be used. In some embodiments, sequence reads meeting minimum coverage of 50% are used as the input for generating consensus sequences and/or a genome construct and for virus detection. The minimum coverage limits may vary from 20% to 100% (e.g., 20, 30, 40, 60, 70, 80, 90 percent).

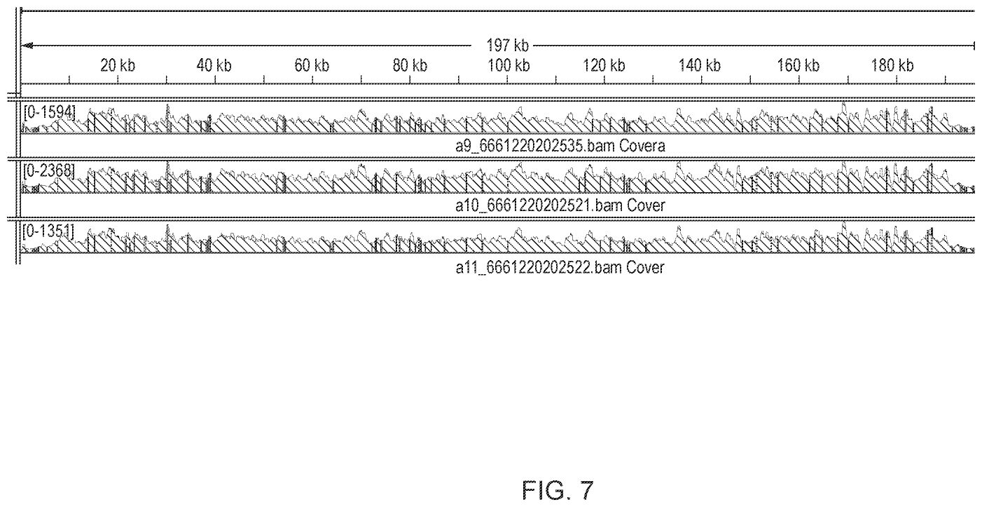

The Adjusted VirSeq Pipeline and the dynamic clinical assay pipeline designs can achieve a better coverage. The better coverage can be achieved through the wet-lab methods and pipelines described in this disclosure. These methods and pipelines involve the precise design of molecule capture and ligation processes, ensuring the sequencing of high-quality molecules. Additionally, the sequencing techniques, such as circular consensus sequencing, further enhance uniform read coverage across the virus genome, enhancing the overall sequencing quality. FIG. 7 is an Integrative Genomics Viewer (IGV) plot with reads per nucleotide on the Y axis and genome coordinates on the X. It can be seen from FIG. 7 of the very even, complete coverage of the MPX genome. Dark vertical bars are locations where variants were detected compared to the reference sequence. Variant detection in introduced later in this section.

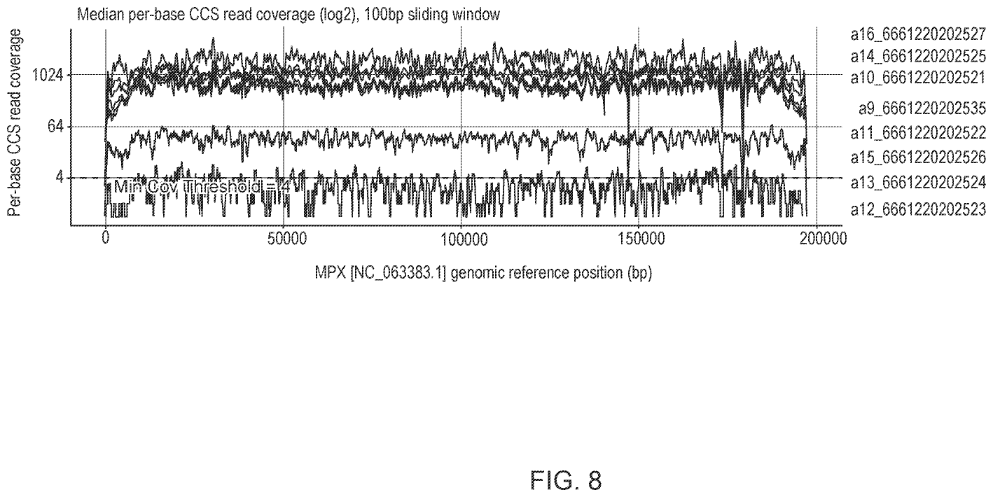

FIG. 8 is a higher resolution graphical representation of the IGV plot shown in FIG. 7 generated using software developed for various clinical samples. Genome coverage and per-base CCS read coverage for eight samples are shown in FIG. 8. When the minimum coverage threshold is predetermined to be four (shown in FIG. 8 as the horizontal dash line), seven of the eight samples achieved the minimum coverage threshold, which shows that the disclosed techniques provide a stable sequence read generation method.

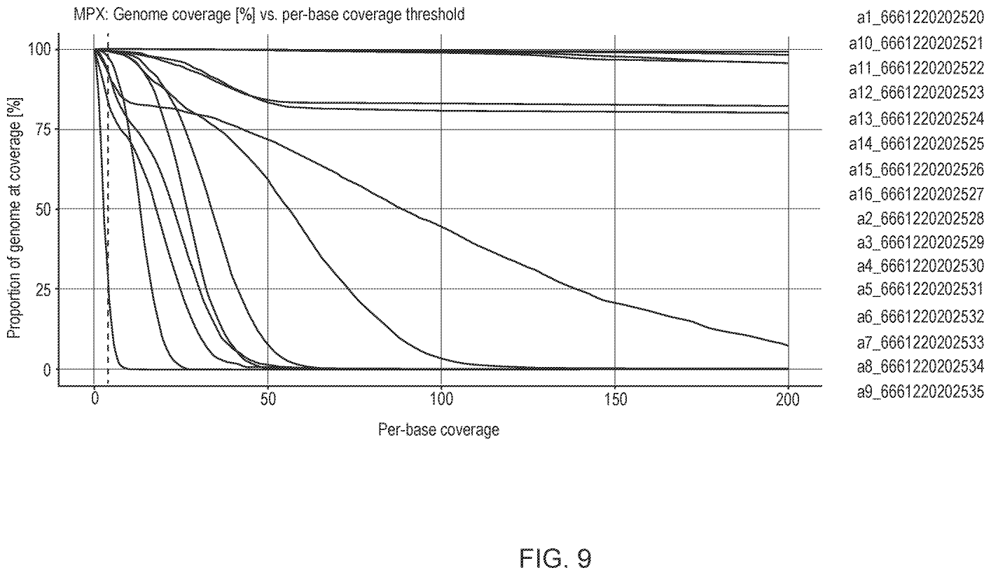

FIG. 9 illustrates an alternative way to demonstrate the coverage of the genome and read depth of various samples and controls. Sequencing data of Sixteen samples are shown in FIG. 9. The vertical dash line marks the minimum coverage threshold (with per base coverage equal to four). As shown in FIG. 9, fifteen out of the sixteen samples achieved the minimum coverage threshold.

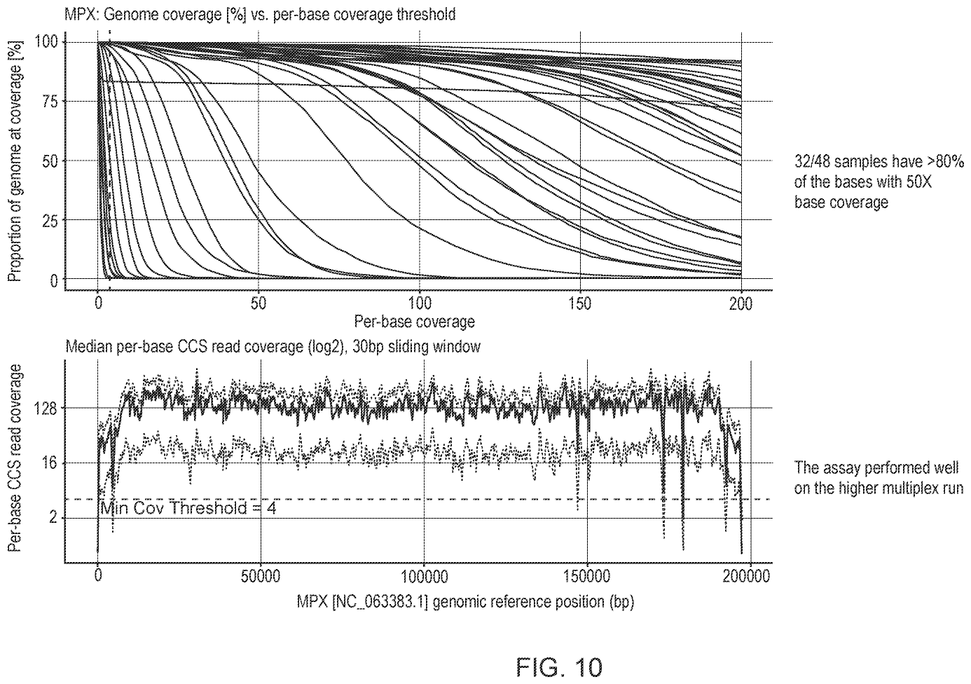

FIG. 10 illustrates genome coverage and per-base coverage in accordance with various embodiments. Top of FIG. 10 shows that 32 out of 48 samples having greater than 80% genome coverage with 50× base coverage. Bottom of FIG. 10 shows individual sample average (solid line) and 25/75 quartiles of coverage (dash lines). FIG. 10 demonstrated that the Adjusted VirSeq Pipeline and the dynamic clinical assay pipeline run well on higher multiplex run. This is accomplished by obtaining and ensuring a greater number of high-quality sequence reads per molecule (e.g., using the precise design of molecule capture and ligation processes). As a result, fewer molecules per sample are required, and more samples can be sequenced simultaneously. This also enhances sequencing efficiency and reduces sequencing expenses.

In some embodiments, the generation of the sequencing file may comprise preprocessing such as demultiplexing. In certain embodiments, preprocessing may comprise generating Circular Consensus Sequence (CCS) BAM files, merging the intermediate BAM files, demultiplexing using to generate individual BAM files corresponding to different barcode combinations, combining demultiplexed output by sample name and/or subject identifier, removing barcodes from sequences and generate individual sample FASTQ files, aligning sequences to barcodes and trimming the barcodes, converting BAM files to FASTQ files and copying FASTQ and CCS BAM files as the sequencing file to a final location.

In some embodiments, the generation of the sequencing file may further comprise filtering sequence reads based on length or quality, aligning the sequence reads to a reference virus genome (e.g., the MPX reference genome). In some embodiments, this alignment is a local alignment performed using tools in a CLC Genomics Server.

The sequencing file may then be used to generate a consensus sequence for each target molecule. In some embodiments, the consensus sequence may be generated using VCFcons (for example, VCFcons v8.5.0). VCFcons is a modified version of VCF, and it is a versatile VCF-based consensus sequence generator for genomes (e.g., small genomes). In some embodiment, a predetermined threshold (e.g., 4) for generating the consensus sequence may be obtained. For example, in certain embodiments, when VCFcons calls a nucleotide identity in a position of the target molecule, it must have at least 4 sequence reads covering that position. In some embodiments, the sequence reads are circular consensus sequencing (CCS) reads. If a nucleotide has less than 4 reads it is reported as an “N” (ambiguous, a non-defined nucleotide) in the consensus sequence. In some embodiments, a nucleotide identity in a position of the target molecule is called when at least 4 sequence reads cover that position and the nucleotide identity in the position is present in at least 50% of the sequence reads that aligned to the position. In some embodiments, a higher percentage (e.g., 60%) or a lower percentage (e.g., 40%) may be used. Methods for generating a consensus sequence for a target molecule are described, for example, in U.S. patent application Ser. No. 17/845,629, the entire content of which is incorporated herein by reference for all purposes.

The sequencing file may also be used to generate a genome construct for each sample. For example, in certain embodiments, when VCFcons calls a nucleotide identity in a position of the genome construct, it must have at least 4 consensus sequences covering that position. If a nucleotide has less than 4 consensus sequences, it is reported as an “N” in the genome construct. In some embodiments, a nucleotide identity in a position of the genome construct is called when at least 4 consensus sequences cover that position and an alternate allele frequency compared to the reference of greater than 50%.

After determining the sequence base compositions, a percentage of non-ambiguous bases may also be determined. In some embodiments, the determination may be based on Seqtk or an alternate algorithm.

Virus detection and/or lineage assignment are determined using techniques disclosed herein (e.g., the disclosed techniques with regard to block 135 of FIG. 1). Both the virus detection and lineage assignment may be determined using the consensus sequences and/or the genome construct and decision rules. In some embodiments, the virus detection is performed using wet-lab means and the lineage assignment is determined using the consensus sequences (or the genome construct) and decision rules. In some embodiments, the decision rules are predetermined (e.g., the decision rules obtained at block 133 of FIG. 1). In other embodiments, the decision rules are determined based on an algorithm or a machine learning model. The machine learning model may be pre-trained using sequences or sequence reads of known lineages of the virus and/or the sequences or sequence reads of the general virus species. In some embodiments, one or more scores are assigned to the consensus sequences and/or the constructed genome using a reference genome of the virus or a virus library as the basis. In some embodiments, the one or more scores are assigned to the consensus sequences and/or the constructed genome using the decision rules.

The one or more scores may be determined based on a distribution of mutations or nucleotide comparisons (e.g., nucleotide substitutions, nucleotide deletions, nucleotide insertions and the like) between the consensus sequences (or the genome construct) and the reference genome or the portion thereof. For example, the distribution shows count of nucleotide substitutions, nucleotide deletions, nucleotide insertions and the like. Different scores may be assigned to different types of mutations or nucleotide comparisons. In some embodiments, different scores are assigned to subtypes in the same type of mutations or nucleotide comparisons. For example, the assigned score for a substitution of “A” to “G” may be different from the assigned score for a substitution of “A” to “C.” In some embodiments, different scores are assigned based on the phylogenetic context (e.g., the evolutionary relationships and lineage classifications) of the mutations or nucleotide comparisons. For example, nucleotide substitutions that are common in specific lineages may receive different scores than those that are rare. In some embodiments, mutations or nucleotide comparisons that lead to changes in the encoded amino acids in protein-coding regions may receive higher scores, especially if the change is known to affect the function of the protein. In some embodiments, a same score is assigned to different types of mutations or nucleotide comparisons.

Once the one or more scores are obtained, they may be used to determine the presence or absence of the virus in the sample and/or its predicted lineage. In some embodiments, when the one or more scores or all of the scores are below a predetermined number or exceed a predetermined range (or threshold), an existing lineage will not be assigned to the virus in the sample. Instead, a new lineage (which is different from existing or known lineages) is recommended and provided to the reporting file.

In some embodiment, NextClade is used for the virus detection and lineage assignment. In certain embodiments, Pangolin may be used to assigns lineages to the consensus sequences or the genome construct. In an embodiment, Pangolin is set so as only to consider genomes that have at least 50% non-ambiguous bases. In certain embodiments, SummaryStat may be used to compile results from NextClade, Pangolin, Seqtk, and other algorithm, platforms, or programs to generate coverage statistics needed for a later quality control (e.g., mean of median amplicon coverage and percent genome coverage). The percent genome coverage may be calculated as the number of non-ambiguous bases (A, T, C, G) divided by the total sequence length, and lineage classifications are aggregated and only samples that produce a NextClade result and Pangolin lineage call are retained for further processing.

For example, to detect a presence or absence of a virus, aligned consensus sequences or the genome construct is compared nucleotide by nucleotide to the reference virus genome or a portion of the reference virus genome. Differences are then scored and/or reported accordingly. In some embodiments, different types of mismatches are scored differently. For example, a nucleotide substitution (a change from one nucleotide to another) may have a higher or lower score than a nucleotide deletion (i.e., a gap). In some embodiments, different nucleotide substitutions may result in different scores. A threshold may be predetermined or based on an algorithm or a machine learning model to determine the presence or absence of the virus based on a total score or a score distribution.

In some embodiments, the presence of the virus in the sample is known and only a predicted lineage of the virus is determined. In some embodiments, the presence of the virus in the sample is determined using PCR or real-time PCR. PCR techniques can be used for detecting viruses. The real-time PCR detection may be performed simultaneously with the dynamic clinical assay pipeline analysis or prior to the dynamic clinical assay pipeline analysis. A diagnostic positive/negative result may be provided to the subject based on the real-time PCR. The diagnostic positive/negative result may also be used to determine whether to input a sample into the dynamic clinical assay pipeline.

Real-time PCR can be used for the detection of viruses. The basic principle of real-time PCR is to amplify and detect specific regions of viral nucleic acid using fluorescent probes or dyes. Using real-time PCR techniques to detect viruses generally comprise: (i) extraction of viral nucleic acid by isolating the viral nucleic acid from the sample; (ii) designing primers and probes targeting conserved regions of the viral genome that are specific to the virus being detected and allow for the amplification and detection of the viral nucleic acid; (iii) preparing a PCR reaction mixture including the primers, probes, and polymerase enzymes, wherein different fluorescent dyes may be added to the reaction mixture; (iv) amplifying using PCR by running in a thermal cycler that cycles through a series of temperature changes to amplify the viral nucleic acid, wherein in each cycle, a fluorescent signal is measured in real-time, allowing for the detection and quantification of the virus; and (v) analyzing data generated during the PCR reaction using specific software to determine the presence and/or amount of viral nucleic acid in the sample. The amount of viral nucleic acid may be expressed as a cycle threshold (Ct) value, which represents the cycle at which the fluorescence signal exceeds a predetermined threshold.

In some embodiments, to assign a lineage, variants of interest may be predetermined, and genomic regions related to the variants of interest may also be determined based on the variants of interest or based on an algorithm or a machine learning model. The genomic regions related to the variants of interest may be a single nucleotide polymorphism (SNP), a consecutive portion in the reference genome, or multiple disconnected portions in the reference genome. One or more scores are determined based on an alignment of the genomic regions of the consensus sequences or the genome construct to the genomic regions of the reference genome or the variants of interest. A threshold may be predetermined or based on an algorithm or a machine learning model to determine the predicted lineage based on the one or more scores or a score distribution.

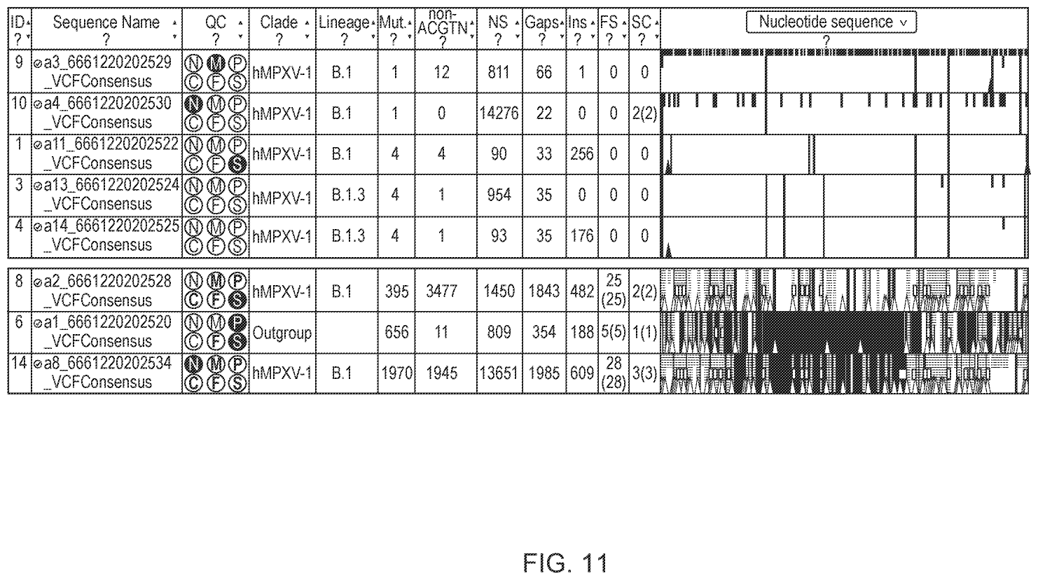

FIG. 11 illustrates exemplary virus calls and lineage assignments in samples. The 5 results (with IDs 9, 10, 1, 3, and 4) in the top table are exemplary circulating strains (under a4 synthetic control) against a published genome isolated in the United States. The 3 results (with IDs 8, 6, and 14) in the bottom table are genomic controls based on previous outbreaks that are no longer in circulation. Black vertical bars show variants to the reference, triangles are insertions, and gray boxes are gaps in the genome, which are expected in synthetic controls.

FIGS. 12A and 12B illustrate a lineage analysis of initial pilot run showing expected assignments for controls and currently circulating strains patient samples. FIG. 12A shows a phylogenetic tree where the samples from the initial pilot run lie in. FIG. 12B is a blowup figure showing all the samples that lie under the B.1 subtree.

FIGS. 13A-13C shows an analysis of larger multiplex run containing more control and patient samples. Expected distribution is shown in FIGS. 13B-13C and it also confirms multiple MPX introductions to the human population as previously reported. Gisaid (database) entry of other lineage detected are shown for comparison. FIG. 13A shows a NextClade linage assignment result.

In some embodiments, the virus detection and the lineage assignment information may be used to dynamically modify the dynamic clinical assay pipeline so that a more efficient and accurate virus detection and lineage assignment results can be achieved in general. For example, the dynamic clinical assay pipeline may be put in clinical use for a period of time and virus detection and lineage assignment information may be collected. Based on the lineage assignment information collected during the period of time, a prevalence of a lineage can be determined, and a reference genome or target molecules may be modified based on the prevalent lineage. In some embodiments, adapters and decision rules may also be modified based on the prevalent lineage. The dynamic clinical assay pipeline may be designed to be automatically modified or updated in real time. The dynamic clinical assay pipeline provides improvement in capturing target molecules and sequencing and also contributes to provide accurate virus detection and lineage assignment results.

### VII. Downstream Applications

Various applications may be applied to the systems and methods disclosed herein. One of the more important applications is for providing a treatment plan or clinical testing protocol for the subject. When a virus is detected in the sample obtained from the subject, a treatment plan or a clinical testing protocol may be automatically generated using a computing system and provided to the subject or a practitioner. The treatment plan or the clinical testing protocol may vary based on the detected virus and/or the predicted lineage. For example, when the virus is a MPX virus or cowpox virus, the treatment plan may comprise administering antiviral medications for the subject.

Additionally, monitoring of new variants and emergence of more pathogenic strains can be crucial. This is because different variants or strains may cause different reactions or lead to different treatments. For instance, there have been cases where some strains of MPX killed up to 10% of patients, while in the latest outbreak, the number of cases was smaller than 1%. The ability of the disclosed techniques to assign a lineage in a sample enables the tracking of virus spread and transmission of different strains. The output from the dynamic clinical assay pipeline can be used to estimate a size of an outbreak by variation in strains, so that a plan to control the spread of the virus or strains can be generated and enacted. The dynamic clinical assay pipeline also allows primer site integrity monitoring. For example, MPX virus/strains drop regions/loci and sometimes the dropped regions/loci are primer sites, and the monitoring the primer site integrity provides feedback to the virus spread and transmission, and the information can also be used to dynamically modify the dynamic clinical assay pipeline.

The disclosed techniques may also be used for designing new assays or machines. The whole or a part of the disclosed techniques may be deployed to the new assay or machine to perform efficient virus detection and/or predicted lineage assignment. In some embodiments, more than one virus may be designed to be detected in the new assay or machine.

### VIII. Examples

The systems and methods implemented in various embodiments may be better understood by referring to the following examples.

### 1. Example 1: Monkeypox Analysis

In an example, 7826 evenly tiled Molecular Loop inversion probes each of that is expected to produce 675 bp fragments of the 200 kb genome of the MPX virus are designed and used in a system described herein. These probe pairs are trimmed from both the ends of the reads using a custom code. In one example, for every sequence that is produced at a sequencing pipeline, 25 bp from both the 5′ and 3′ end of the sequence are trimmed off.

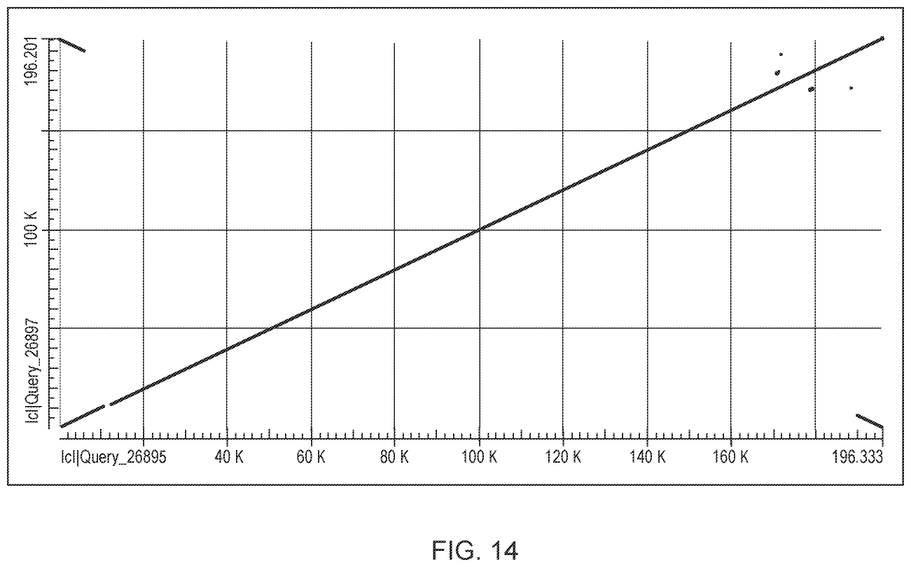

FIG. 14 demonstrates a comparison between the sequenced synthetic control and the independently obtained sequencing data, highlighting concordant regions with linear relationships to mismapping. The synthetic control is genomes with known truth supplied by manufacturers. It is compared to independently obtained sequencing data. A dot means that both sequences have identical bases at the genomic position. As shown in FIG. 14, the highly repetitive repeats at the 5′ and 3′ genomic ends are expected to be dual mapped.

### 2. Example 2: SARS-CoV-2 Analysis

The Labcorp VirSeq SARS-CoV-2 NGS Test can be performed at laboratories designated by Labcorp that are certified under the Clinical Laboratory Improvement Amendments of 1988 (CLIA), 42 U.S.C. § 263a, and meet the requirements to perform high complexity tests as described in the Labcorp VirSeq SARS-CoV-2 NGS Test Standard Operating Procedure that was reviewed by the FDA under Emergency Use Authorization (EUA).

### Intended Use

The Labcorp VirSeq SARS-CoV-2 NGS Test is a next generation sequencing (NGS) test on the PacBio Sequel II sequencing system intended for the identification and differentiation of SARS-CoV-2 Phylogenetic Assignment of Named Global Outbreak (PANGO) lineages, when clinically indicated, from SARS-CoV-2-positive samples identified using Labcorp's COVID-19 RT-PCR Test or Labcorp SARS-CoV-2 & Influenza A/B Assay. Testing is limited to laboratories designated by Labcorp that are certified under Clinical Laboratory Improvement Amendments of 1988 (CLIA), 42 U.S.C. § 263a, and meet requirements to perform high complexity tests The Labcorp VirSeq SARS-CoV-2 NGS Test is intended to be used in conjunction with patient history and other diagnostic information, when clinically indicated, i.e., in situations where results may aid in determining appropriate clinical management. Results of this test are intended to be interpreted by the ordering health care professional. The test is not intended for use as an aid in the primary diagnosis of infection with SARS-CoV-2 or to confirm the presence of SARS-CoV-2 infection, and it is not intended for identification of specific SARS-CoV-2 genomic mutations. Results should not be used as the sole basis for treatment or other patient management decisions.

The Labcorp VirSeq SARS-CoV-2 NGS Test is intended for use by qualified clinical laboratory personnel specifically instructed and trained in the operation of the PacBio Sequel II sequencing system and next generation sequencing workflows as well as in vitro diagnostic procedures. The Labcorp VirSeq SARS-CoV-2 NGS Test is only for use under the Food and Drug Administration's Emergency Use Authorization.

### Device Description and Test Principle

The Labcorp VirSeq SARS-CoV-2 NGS Test is a PacBio Sequel II-based whole genome sequencing assay used for the determination of PANGO lineage from extracted RNA of SARS-CoV-2 positive samples identified using Labcorp's COVID-19 RT-PCR Test or Labcorp SARS-CoV-2 & Influenza A/B Assay. The SARS-CoV-2 probe set used in this assay contains ˜1000 tiled Molecular Loop Inversion Probes (MIPS) designed to amplify RNA that has been reverse transcribed to cDNA from 99.6% of the SARS-CoV-2 genome with most bases covered by 22 MIPs. The product synthesized in-between the MIPS is enriched and has sample specific molecular barcodes added via amplification followed by sequencing.

Residual total nucleic acid extract from SARS-CoV-2 positive RT-PCR diagnostic testing samples with N1 target Cycle Threshold (Ct) values<31 are tested with the Labcorp VirSeq SARS-CoV-2 NGS Test. Residual nucleic acid extract can be stored at −20° C. for up to 30 days through up to 2 freeze-thaw cycles. Residual total nucleic acid extract is transferred into a 96 well plate containing only positive RT-PCR diagnostic testing samples using Hamilton Microlab STAR. Samples are then aliquoted into a sequencing run plate of 94 samples with one water non-template control (NTC) and one positive control. Eight plates, or 752 specimens, are processed in one production batch.

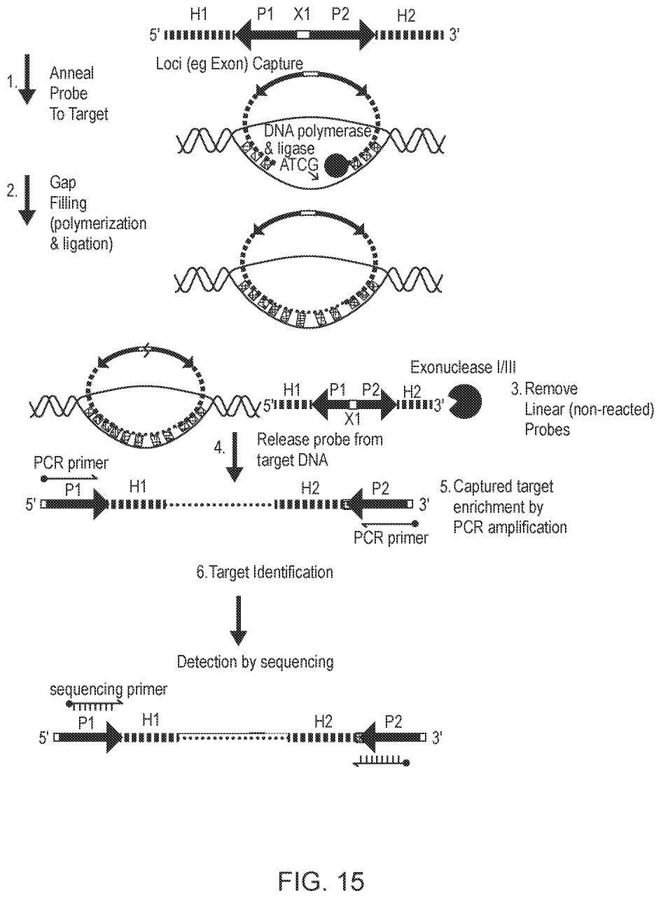

A custom Molecular Loop SARS-CoV-2 Capture Kit is used to prepare samples to be sequenced on the PacBio Sequel II instrument. First, a reverse transcriptase enzyme provided by Thermo Fisher synthesizes cDNA from RNA. SARS-CoV-2 cDNA is then used as a target for hybridization of molecular loop probes (FIG. 15, Step 1) (Information regarding FIG. 15 can be found in FDA website, EMERGENCY USE AUTHORIZATION (EUA) SUMMARY Labcorp VirSeq SARS-CoV-2 NGS Test, downloaded on Mar. 22, 2023). Molecular loop probes are tiled across 99.6% of the 30 kb SARS-CoV-2 viral genome and consist of two binding sites 600 bp apart. After binding, the 600 bp region in-between the two probes are synthesized with DNA polymerase and ligated to form a closed molecule (FIG. 15, Step 2). Non-binding or incomplete loops remain linear molecules and are removed with exonuclease digestion (FIG. 15, Step 3). Circular molecules are then released from the template cDNA (FIG. 15, Step 4) and enriched via amplification with a 3′ molecular loop specific M13 universal sequence and 5′ sample specific barcodes (FIG. 15, Step 5). Samples are then pooled by equal volume and library prepped for PacBio sequencing. Library preparation entails DNA damage repair, ligation of sequencing adapters, non-ligated product removal by enzymatic digestion, and bead purification. Libraries are then sequenced on a PacBio Sequel II with 15-hour movies (FIG. 15, Step 6).

After sequencing, PacBio SMRT LINK software and custom molecular loop processing scripts are used to generate the FASTQ files for each sample. FASTQs are analyzed using a genome analysis pipeline implemented in the CLC genomics server version 9.1.1. This workflow starts with a sample-level fastq file, trims primers, and uses Minimap2 to align to the SARS-CoV-2 reference genome (“NC_045512v2”) to generate a bam file of the alignment. A consensus sequence for each sample is generated using VCFcons (v8.5.0). When VCFcons calls a nucleotide sequence for genome construction it must have at least 4 circular consensus sequencing (CCS) reads covering that base pair and an alternate allele frequency compared to the reference of greater than 50%. If a nucleotide is covered by less than 4 reads it is reported as ambiguous (N) in the consensus sequence. The lineages for individual samples are then assigned using the consensus sequence as input to the PANGOLIN (v3.1.20) analysis package. Lineage results are released for samples with at least 90% genome coverage and whose overall genomic coverage is greater than 10 CCS reads. The overall genomic coverage of a sample is defined as the mean of median read coverage across 29 (˜1 kb length) consecutive regions which spans the whole viral genome.

Instruments Used with the Test

The Labcorp VirSeq SARS-CoV-2 NGS Test is to be used with the Pacific Bioscience Sequel II sequencing instrument, the Mantis liquid handler and the Labcorp VIRSEQ Analysis Pipeline for sequence analysis and lineage determination. The instruments and reagents required in order to perform the Labcorp VirSeq SARS-CoV-2 NGS Test are presented in Table 1.

TABLE 1
Reagent and Special Instrument Requirements
SPECIAL INSTRUMENTS

| Equipment | Vendor | Catalog # |
|---|---|---|
| ProFlex PCR System | Thermo | 4483636 |
| 10, 20, 200 and 1000 μL pipettors | Rainin | BT-10, BT-2003100 |
| Quantus | Promega | E6150 |
| DynaMagTM Magnet | Life Technologies | 12331D |
| Microcentrifuge | Thermo Fisher | 540394 |
| Vortex | Thermo Fisher | various |
| Sequel II | Pacific Bioscience | N/A |
| Mantis | Formulatrix | N/A |
| Liquidator | Rainin | 17010335 |

REAGENTS

| Reagent | Vendor | Catalog # |
|---|---|---|
| AMPure PB Beads | Pacific Bioscience | 100-262-900 |
| SMRT CELL 8M TRAY EA | Pacific Bioscience | 101-389-001 |
| Sequel II Sequencing Plate 2.0 | Pacific Bioscience | 101-826-100 |
| SMRTBELL ENZYME CLEAN UP | Pacific Bioscience | 101-746-400 |
| KIT | | |
| SuperScriptTM VILOTM Master Mix | Thermofisher | 11755500 |
| SARS-COV2 CAPTURE KIT CUST | Molecular Loop | ML5902-CP-LCPB1 |
| Asymmetric Barcodes | IDT | 14046396 |
| 200 proof ethanol | Pharmco-Aaper | 111000200 |
| Molecular Biology Grade Water | Krackler | 45-W4502-1L |
| SMRTbell Express Template Prep kit | Pacific Biosciences | 100-938-900 |
| 2.0 | | |
| Sequel II Binding Kit 2.1 | Pacific Biosciences | 100-820-500 |
| Sequel II DNA Internal Control | Pacific Biosciences | 101-717-600 |
| Complex 1.0 | | |
| Quantifluor dsDNA System | Promega | E2670 |
| Sequel Smart Cell Oil | Pacific Biosciences | 100-621-300 |
| Twist Synthetic RNA Controls | Twist | 102019, 102862, 103909, 104043, |
| | | 104044, 104338, 104529, |
| | | 104530, 104532, 104533 |

SOFTWARE

| Software name | Developer | Version |
|---|---|---|
| Smrtlink | Pacific Bioscience | v9.0 |
| Anaconda | Anaconda Inc. | v2019.03 (conda v4.11.0) |
| Python | Python Software | v3.7.6, v3.9 |
| | Foundation | |
| Lima | Pacific Bioscience | v1.11.0 |
| Pbmm2 | Pacific Bioscience | v1.2.1 |
| BamTools | MIT | v2.4.1 |
| CLC Genomics Grid Worker | Qiagen | 9.1.1 |
| CLS-plugin | | 1.3.5 |
| Miniconda2 | Anaconda Inc. | conda v4.8.3 |
| Pangolin | COVID-19 | v3.1.20 |
| | Genomics UK | |
| | Consortium | |
| NextClade | NextStrain project | v1.3.0 |
| Seqtk | MIT | v1.2 |
| Minimap2 | Dana-Farber | v2.17-r941 |
| | Cancer Institute | |
| Samtools | MIT | v1.3.1 |
| VCFCons | | v8.5.0 |
| Hamilton Venus | Hamilton Company | v4.0 |

Designated laboratories will receive an FDA accepted instrument qualification protocol included as part of the Labcorp VirSeq SARS-CoV-2 NGS Test Standard Operating Procedure (SOP) and will be directed to execute the protocol prior to testing clinical samples. Designated laboratories must follow the authorized SOP, which includes the instrument qualification protocol, as per the letter of authorization.

Controls to be Used with the Test

External Positive Control: An external positive control will be added to each plate of 94 patient samples. The control consists of one of ten Twist synthetic SARS-CoV-2 RNA controls with predetermined PANGO Lineage designation. The lineage identification result of the external positive control will be compared to its known PANGO Lineage designation.

External Negative Control: An external non-template control (NTC) is needed to ensure master mix contamination events are not present on the given amplification plate. The control consists of molecular grade water added to the A1 position of every 96 well plate before sample addition. The NTC is then transferred along with positive samples to the sequencing run plate and taken through sequencing and quality control (QC) analysis.

Other Controls: Lineage identification results are only released for samples with at least 90% genome coverage and whose overall genomic coverage is greater than 10 CCS reads.

### Interpretation of Results

All test controls should be examined prior to interpretation of patient results. If the controls are not valid, the patient results cannot be interpreted.

### 1) Labcorp VirSeq SARS-CoV-2 NGS Test Controls—Positive:

After sequencing, the lineage designation of a given plate's positive control will be compared to the known lineage designation of the positive control. A positive control sample will be considered a failure if its assay determined lineage differs from its known lineage. Samples on plates with failed positive external control are re-analyzed one additional time with the assay using material from the original total nucleic acid extract. If a sample's residual total nucleic acid is depleted, it is reported as a failed sample.

### 2) Labcorp VirSeq SARS-CoV-2 NGS Test Controls—NTC:

After sequencing, the overall genomic coverage is calculated for the NTC samples. A NTC sample will be considered a failure if it has overall genomic coverage greater than 10 CCS reads. Samples on plates with failed negative external control are re-analyzed one additional time with the assay using the original total nucleic acid extract. If a sample's residual total nucleic acid is depleted, it is reported as a failed sample.

TABLE 2
Expected Results for the Labcorp VirSeq
SARS-CoV-2 NGS Test External Controls

| External Control | | |
|---|---|---|
| Type | Passing Criteria | Action For Control Failure |
| Positive External | Assay determined | Plates with failed positive |
| Control | lineage designation | external controls are re- |
| | is concordant with | analyzed one additional time |
| | known lineage | with the assay using the |
| | designation. | original total nucleic acid |
| | | extract. If a sample's residual |
| | | total nucleic acid is depleted, it |
| | | is reported as a failed sample. |
| Negative External | Overall genomic | Plates with failed negative |
| Control | coverage is greater | external controls are re- |
| | than 10 CCS reads. | analyzed one additional time |
| | | with the assay using the |
| | | original total nucleic acid |
| | | extract. If a sample's residual |
| | | total nucleic acid is depleted, it |
| | | is reported as a failed sample. |

### 3) Examination and Interpretation of Patient Sample Results:

Assessment of Labcorp VirSeq SARS-CoV-2 NGS Test results are performed after external control analysis and removal of any samples on a plate with a failed external control. The overall genomic coverage and percent genome coverage is calculated for each patient sample. Lineage results are released for samples with at least 90% genome coverage and overall genomic coverage greater than 10 CCS reads.

The interpretation and reporting of clinical specimens are summarized in Table 3.

TABLE 3
Result Interpretation for Patient Samples

| SAMPLE | EXTERNAL | | |
|---|---|---|---|
| LEVEL QC | CONTROLS | PANGOLEARN | |
| RESULTS | RESULTS | OUTPUT | ACTION |
| Pass | Pass | Lineage call | Report results to sender and |
| | | | appropriate public health |
| | | | authorities. |
| | | None | Sample is repeated**. If no |
| | | | residual sample, it will be |
| | | | reported as failed*. |
| | Fail | Not Applicable | Plate is repeated. If no residual |
| | | | sample, it will be reported as |
| | | | failed*. |
| | | Not Applicable | Plate is repeated. If no residual |
| | | | sample, it will be reported as |
| | | | failed*. |
| Fail | Pass | Not Applicable | Sample is reported as failed*. |
| | Fail | Not Applicable | Plate is repeated. If no residual |
| | | | sample, it will be reported as |
| | | | failed*. |

*Failed samples will be reported as: “No lineage was able to be determined. SARS-CoV-2 virus detected, but genome sequencing was unsuccessful. No lineage information can be reported.”
**Individual failing samples will only be repeated once and after a second failure will be reported as described above for a failed sample.

### Performance Evaluation

1. Device Tolerance:

Residual nucleic acid extracts from 8,815 respiratory specimens which tested positive for SARS-CoV-2 by the EUA200011 authorized Labcorp COVID-19 Test across 3 sites were sequenced with the Labcorp VirSeq SARS-CoV-2 NGS Test. The number of samples that produced genomic sequences that passed both of the following quality control criteria were determined for different N1 CT value categories.
Greater than 90% genome coverage when compared to the SARS-CoV-2 reference genome (“NC_045512v2”)
Overall genomic coverage is greater than 10 CCS reads.

TABLE 4
Tolerance Study Result

| | | Number of | Percent (%) of |
|---|---|---|---|
| Cycle | Number of | Specimen | Specimen Passing |
| Threshold | Specimen | Passing Both | Both QC Criteria |
| (CT) | Tested | QC Criteria | (95% CI) |
| <16 | 26 | 23 | 88.5% (69.8%-−97.6%) |
| 16-17 | 39 | 36 | 92.3% (79.1%-−98.4%) |
| 17-18 | 123 | 111 | 90.2% (83.6%-−94.9%) |
| 18-19 | 318 | 291 | 91.5% (87.9%-−94.3%) |
| 19-20 | 415 | 383 | 92.3% (89.3%-−94.7%) |
| 20-21 | 661 | 611 | 92.4% (90.1%-−94.3%) |
| 21-22 | 814 | 757 | 93.0% (91.0%-−94.7%) |
| 22-23 | 834 | 767 | 92.0% (89.9%-−93.7%) |
| 23-24 | 913 | 831 | 91.0% (89.0%-−92.8%) |
| 24-25 | 876 | 759 | 86.6% (84.2%-−88.8%) |
| 25-26 | 800 | 625 | 78.1% (75.1%-−80.9%) |
| 26-27 | 685 | 437 | 63.8% (60.1%-−67.4%) |
| 27-28 | 682 | 330 | 48.4% (44.6%-−52.2%) |
| 28-29 | 582 | 160 | 27.5% (23.9%-−31.3%) |
| 29-30 | 518 | 87 | 16.8% (13.7%-−20.3%) |
| 30-31 | 529 | 140 | 7.6% (5.5%-−10.2%) |

2. Precision (Repeatability): Intra-Assay

Intra-assay repeatability was assessed by testing 11 nucleic acid samples in triplicate. The intra-assay repeatability study assessed the ability of the assay to accurately detect lineages on the replicates of the same samples during one assay run. The N1 CT value of the samples tested ranges from 19.2 to 22.96.

Lineage designation of all 11 samples were concordant across the three replicates with all replicates meeting QC criteria.

3. Precision (Reproducibility): Inter-Assay

Inter-assay reproducibility was assessed by testing the same nucleic acid samples assessed in the repeatability study in triplicate over three assay runs. One of the 11 samples tested in the repeatability study was unintentionally excluded from the final run. Sequencing runs were performed by 6 different technologists using 3 different lots of SMRT cells and 2 lots of sequencing reagents. The third run was 3 weeks from the first run and was multiplexed at ˜4× lower sample concentration than the previous 2 runs.

TABLE 5
Reproducibility Study Description

| | | | Sequencing |
|---|---|---|---|
| Run ID | Time | Personnel | Reagent Lots |
| Run 1 | Jul. 7, 2021 | Technologist | 1 |
| | | 1, 2, 3 and 4 | |
| Run 2 | Jul. 14, 2021 | Technologist | 2 |
| | | 2, 4 and 5 | |
| Run 3 | Jul. 28, 2021 | Technologist 5 and 6 | 2 |

Lineage designations for 9 out of 10 samples were concordant across all 3 runs. The discordant sample did not pass the overall genome coverage QC criteria in Run 2 and Run 3.

4. Sample Stability (Freeze-Thaw)

Stability of patient samples under recommended storage conditions was assessed in the sample stability study. After Nucleic Acid Amplification (NAA) diagnostic testing with Labcorp COVID-19 RT-PCR TEST, extracted nucleic acid was shipped on dry ice to the testing laboratory and stored at −20° C. before sequencing.

Twelve samples comprising of Alpha, Beta and Delta Variants of Concerns (VOCs) were sequenced initially. These samples were re-sequenced after 5 weeks of −20° C. storage that entailed 3 freeze-thaw cycles. 11 out of the 12 samples produced concordant results between initial, and repeat, testing. The only discordant result was determined to be due to mechanical errors and not related to sample stability. The result of the study supports the stability of the Labcorp VirSeq SARS-CoV-2 NGS Test for up to 2 freeze-thaw cycles when samples are stored at 20° C. for up to 30 days.

5. Concordance

Lineage designation results produced by the Labcorp VirSeq SARS-CoV-2 NGS Test (PacBio Molecular Loop Sequencing) were directly compared to lineage designation results generated by Illumina COVIDSeq (RUO-surveillance protocol with v3 primer pool) as well as PacBio Amplicon Sequencing. The analysis included the following samples.
Illumina COVIDSeq (RUO) Samples
   93 Negative (NAA) samples
   72 SARS-CoV-2 samples sequenced in Winter 2020
   29 samples previously sequenced at Center for Molecular Biology and Pathology (CMBP)
   50 samples previously sequenced at DNA Identification
PacBio Amplicon Sequenced Samples
   122 SARS-CoV-2 samples that were amplicon sequenced at 90% coverage.

### Illumina COVIDSeq (RUO) Comparison:

93 samples previously determined to be negative by Labcorp COVID-19 RT-PCR diagnostic tests were sequenced in duplicate using the Labcorp VirSeq SARS-CoV-2 NGS Test and Illumina COVIDSeq (RUO). Out of the 93 samples tested, one sample resulted in reportable SARS-CoV-2 genomes using Illumina COVIDSeq (RUO) alone, and another sample resulted in reportable SARS-CoV-2 genomes for both Illumina COVIDSeq (RUO) and the Labcorp VirSeq SARS-Cov-2 NGS Test. Further investigation revealed both samples were positive for SARS-CoV-2 by the Labcorp COVID-19 RT-PCR Test and mistakenly included in the validation. The final concordance between Illumina COVIDSeq (RUO) and Labcorp VirSeq SARS-CoV-2 NGS Test for the 91 true negative samples was 100%.

From the 72 samples sequenced in winter 2020 using Illumina COVIDSeq (RUO), 51 samples produced reportable results when using the Labcorp VirSeq SARS-CoV-2 NGS Test. Out of the 51 reportable results, all lineage designation results were 100% concordant with the Illumina COVIDSeq (RUO) output.

TABLE 6
Comparison of PANGO Lineage Designation between Illumina
COVIDSeq (RUO) and Labcorp VirSeq SARS-CoV-2 NGS
Test for Winter 2020 circulating lineages.

| | | Concordant | Reportable |
|---|---|---|---|
| Samples | Reportable | Reportable | Result |
| Tested | Result | Result | Concordant % |
| 77 | 51 | 51 | 100% |

A total of 7 of the 79 samples were lost in transit or did not produce a valid result with Illumina COVIDSeq (RUO). 72 of the 79 samples collected at CMBP and DNA Identification were successfully sequenced with both Illumina COVIDSeq (RUO) and the Labcorp VirSeq SARS-CoV-2 NGS Test. All 66 out of these 72 samples that passed sequences QC criteria for the Labcorp VirSeq SARS-CoV-2 NGS Test have concordant lineage designation result with Illumina COVIDSeq (RUO).

TABLE 7
Comparison of PANGO lineage designation between Illumina
COVIDSeq (RUO) and Labcorp VirSeq SARS-CoV-2 NGS
Test for Summer 2021 circulating lineages.

| | | Concordant | Reportable |
|---|---|---|---|
| Samples | Reportable | Reportable | Result |
| Tested | Result | Result | Concordant % |
| 72 | 66 | 66 | 100% |

### PacBio Amplicon-Based Sequencing Comparison:

122 samples originally sequenced using a PacBio amplicon-based approach were reprocessed using the Labcorp VirSeq SARS-CoV-2 NGS Test in duplicate. Each of the 122 samples with >90% coverage via amplicon sequencing, were tested in duplicate with the Labcorp VirSeq SARS-CoV-2 NGS Test; therefore, a total of 244 results were produced. Of the 244 Labcorp VirSeq SARS-CoV-2 NGS Test lineage determination results, 234 results produced genomes that passed both QC metrics. Among these 234 results, 225 were concordant with the original PacBio amplicon assay lineage designation. When compared to the PacBio amplicon sequencing lineage designation result, 96% samples produced concordant lineage identification results.

TABLE 8
Comparison of PANGO lineage designation between PacBio Amplicon
Sequencing and Labcorp VirSeq SARS-CoV-2 NGS Test

| | | Concordant | Reportable |
|---|---|---|---|
| Number of | Reportable | Reportable | Result |
| Results | Result | Result | Concordant % |
| 244 | 234 | 225 | 96% |

6. Reference Sample Testing

Heat-inactivated SARS-CoV-2 samples from B.1.1.7 (VR-3326HK), Hong Kong/VM20001061 and Italy-INMI1 lineages, characterized by ATCC, were used in this evaluation. Sequencing error associated with the Labcorp VirSeq SARS-CoV-2 NGS Test was evaluated by comparing all mutations identified in the consensus sequences produced by the Labcorp VirSeq SARS-CoV-2 NGS Test analysis pipeline to the published ATCC reference sequences. The results are shown in the following table:

TABLE 9
Number of nucleotide mismatches between ATCC reference sequences
and Labcorp VirSeq SARS-CoV-2 NGS Test generated sequences

| | | | | | Percent |
|---|---|---|---|---|---|
| | # Mutation | # Reference | #Additional | | (%)Mismatch |
| | Observed in | Mutations | Mutations | | Between VirSeq |
| | Reference | Identified in | Identified in | | Consensus |
| | Sequence | the Virseq | the Virseq | Nucleotide | Sequence and |
| ATCC | (Reference | Consensus | Consensus | Positions | Reference |
| strain* | Mutation) | Sequence | Sequence | Sequenced | Genome |
| b1117 | 35 | 33 | 2 | 29683 | 0.013% |
| HK | 15 | 12 | 0 | 29780 | 0.010% |
| ITLY | 5 | 3 | 1 | 29979 | 0.010% |
| Total | 55 | 48 | 3 | 89442 | 0.012% |

*b1117, B.1.1.7 (VR-3326HK); HK, Hong Kong/VM20001061; ITLY, Italy-INMI1

Overall, an average of 0.012% sequence differences were observed between the reference sequence and the consensus sequence produced by the Labcorp VirSeq SARS-CoV-2 NGS Test.

7. Simulation Study

A simulation study was conducted to assess the performance of the Labcorp VirSeq SARS-CoV-2 NGS Test to identify samples with PANGO lineages not tested in the concordance and analytical studies.

A sequencing error model that simulates how sequencing error and ambiguous nucleotides are randomly introduced by the Labcorp VirSeq SARS-CoV-2 NGS Test into the sequenced genome, was estimated based on the sequencing results of 760 clinical samples. The model estimated that the Labcorp VirSeq SARS-Cov-2 NGS Test, on average, results in 1 sequencing error per 33 SARS-CoV-2 genomes sequenced and 3,369 ambiguous nucleotides per SARS-CoV-2 genome sequenced.

### Variant of Concern/Variant of Interest Simulation

A total of 23,400 reference sequences were downloaded from GISAID with known Pango-lineage designation representing 234 lineages (100 reference sequence per lineage). The sequencing error model was used to introduce sequencing errors into these 23,400 reference sequences to simulate the hypothetical sequence output of the assay for these genomes. Each of these 23,400 sequences were used to produce multiple simulated sequences. The PANGO Lineages of these simulated sequences are identified with the lineage identification software (PANGOLIN v3.1.20) used in the Labcorp VirSeq SARS-CoV-2 NGS Test. The lineage identification results of each simulated sequence were compared to the known PANGO Lineage Designation of the reference sequence used to produce the simulated sequence. The concordance results of simulated sequences with genome coverage of 90%, 95% and 99% are shown in the following table:

TABLE 10
In silico performance of Labcorp VirSeq SARS-
CoV-2 NGS Test for 234 VOC/VOI lineages.

| Genome | #Sequences | # Concordance | % Concordance |
|---|---|---|---|
| Coverage | Tested | Sequence | Sequence (95% CI) |
| 90% | 17538 | 16762 | 95.58% (95.26%-95.86%) |
| 95% | 16274 | 15739 | 96.71% (96.42%-96.97%) |
| 99% | 11169 | 10929 | 97.85% (97.56%-98.10%) |

100% (95% CI 99.55%-100.00%) of the 770 simulated Omicron sequences were accurately identified on a sub-lineage level (BA.1, BA.2 and BA.3).

### Time Period Simulation

A total of 10,000 high quality SARS-CoV-2 sequences were randomly sampled from GISAID. A total of 140,000 reference sequences were downloaded from GISAID with known PANGO lineage designation. The sequencing error model was used to introduce sequencing errors into these 140,000 reference sequences to simulate hypothetical sequence output of the assay for these genomes. Each of the 140,000 sequences were used to produce multiple simulated sequences. The PANGO lineages of these simulated sequences are identified with the lineage identification software (PANGOLIN v3.1.20) used in the Labcorp VirSeq SARS-CoV-2 NGS Test. The simulated lineage identification results are compared to the known PANGO lineage designation of the reference sequence used to produce the simulated sequence. The concordance results of simulated reads with genome coverage of 90%, 95% and 99% are shown in the following table:

TABLE 11
In silico performance of Labcorp VirSeq SARS-CoV-2
NGS Test for 140,000 sequences submitted to GISAID.

| Genome | #Sequences | # Concordance | % Concordance |
|---|---|---|---|
| Coverage | Tested | Sequence | Sequence (95% CI) |
| 90% | 105930 | 102270 | 96.54% (96.43%-96.65%) |
| 95% | 96333 | 94222 | 97.81% (97.71%-97.89%) |
| 99% | 45929 | 45182 | 98.37% (98.25%-98.48%) |

All of the 43,328 simulated Omicron sequences were accurately identified as Omicron sequences. The sub-lineage concordance rates for the simulated Omicron sequences are shown in the following table:

TABLE 12
In silico performance of Labcorp VirSeq SARS-CoV-2
NGS Test for 43,328 simulated Omicron sequences.

| Sub | Genome | # Sequences | # Concordance | Percent (%) Concordance | |
|---|---|---|---|---|---|
| Lineage | Coverage | Tested | Sequence | Sequence (95% CI) | |
| BA.1 | 90% | 7466 | 7465 | 99.98% | (99.92%-99.99%) |
| | 95% | 6791 | 6791 | 100.00% | (99.94%-100.00%) |
| | 99% | 3421 | 3421 | 100.00% | (99.88%-99.99%) |
| BA.1.1 | 90% | 7167 | 7029 | 98.07% | (97.73%-98.36%) |
| | 95% | 6520 | 6479 | 99.37% | (99.14%-99.53%) |
| | 99% | 3523 | 3523 | 100.00% | (99.89%-100.00%) |
| BA.2 | 90% | 3429 | 3429 | 100.00% | (99.88%-100.00%) |
| | 95% | 3161 | 3161 | 100.00% | (99.87%-100.00%) |
| | 99% | 1833 | 1833 | 100.00% | (99.79%-100.00%) |
| BA.3 | 90% | 7 | 7 | 100.00% | (64.56%-100.00%) |
| | 95% | 5 | 5 | 100.00% | (56.55%-100.00%) |
| | 99% | 5 | 5 | 100.00% | (56.55%-100.00%) |

### IX. Computing Systems and Additional Considerations

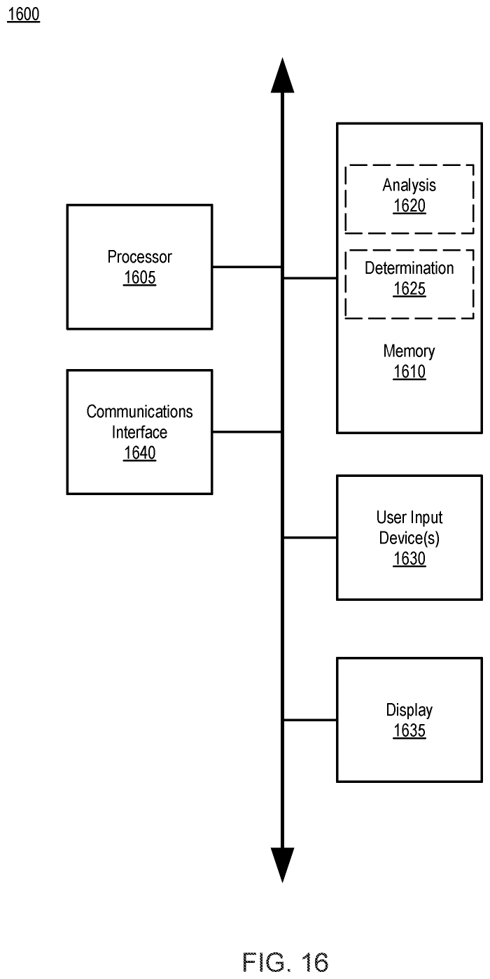

FIG. 16 illustrates an example computing device 1600 suitable for use with systems and methods for determining a presence or absence of a virus and its predicted lineage in a sample obtained from a subject according to this disclosure. The example computing device 1600 includes a processor 1605 which is in communication with the memory 1610 and other components of the computing device 1600 using one or more communications buses 1615. The processor 1605 is configured to execute processor-executable instructions stored in the memory 1610 to perform one or more methods for determining a presence or absence of a virus and its predicted lineage in a sample obtained from a subject according to different examples, such as part or all of the exemplary system 100, process 200 or 300 described above with respect to FIGS. 1-3.

The computing device 1600, in this example, also includes one or more user input devices 1630, such as a keyboard, mouse, touchscreen, microphone, etc., to accept user input. The computing device 1600 also includes a display 1635 to provide visual output to a user such as a user interface. The computing device 1600 also includes a communications interface 1640. In some examples, the communications interface 540 may enable communications using one or more networks, including a local area network (“LAN”); wide area network (“WAN”), such as the Internet; metropolitan area network (“MAN”); point-to-point or peer-to-peer connection; etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

Specific details are given in the above description to provide a thorough understanding of the embodiments. However, it is understood that the embodiments can be practiced without these specific details. For example, circuits can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine-readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium,” “storage,” or “memory” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

What is claimed is:

1. A method comprising:
obtaining nucleic acid from a sample that was obtained from a subject;
capturing a target molecule in the nucleic acid using a molecular inversion probe under hybridization conditions;
amplifying the target molecule using polymerase chain reaction (PCR) to obtain a plurality of amplified molecules;
for each molecule in the plurality of amplified molecules,
ligating an adapter to each end of the molecule to create a circular molecule; and
sequencing the circular molecule to obtain sequence reads;
generating, using a computing system, a sequencing file comprising the sequence reads of each molecule in the plurality of amplified molecules and a position of each sequence read in a reference genome of a virus by aligning the sequence read to the reference genome of the virus; and
generating, using the computing system and the sequencing file, a reporting file for the subject, wherein the reporting file comprises a predicted lineage of the virus in the sample, wherein the generating comprises:
generating a consensus sequence for the target molecule based on sequence reads of each molecule in the plurality of amplified molecules, wherein a nucleotide identity is assigned to a position in the consensus sequence if at least a predetermined number of the sequence reads has the nucleotide identity in the position, and wherein an “N” is assigned to a position in the consensus sequence if less than a predetermined number of the sequence reads has the nucleotide identity in the position;
determining one or more scores of the consensus sequence based on the reference genome of the virus or a library of the virus, wherein the one or more scores are determined based on a distribution of mutations of the virus; and
determining the predicted lineage of the virus in the sample based on the one or more scores of the consensus sequence.

2. The method of claim 1, wherein the reporting file further comprises a presence or absence of the virus in the sample, and wherein the presence or absence of the virus in the sample is determined using real-time PCR (RT-PCR) or based on the one or more scores of the consensus sequence.

3. The method of claim 1, wherein the virus is monkeypox (MPX) virus.

4. The method of claim 1, wherein the molecular inversion probe consists of two binding sites about 600-700 bp apart.

5. The method of claim 1, wherein the sequencing file is a Binary Alignment Map (BAM) file, and the reporting file is a Variant Call Format (VCF) file.

6. The method of claim 1, further comprising providing a treatment plan or clinical testing protocol for the subject, wherein the treatment plan comprises administering antiviral medications for the subject.

7. The method of claim 1, further comprising:
obtaining a prevalent lineage of the virus for a subject population, wherein the prevalent lineage is determined based on the predicted lineage of the virus in the sample;
updating the molecular inversion probes to capture the target molecule, wherein the target molecule is specific to the prevalent lineage;
updating the adapters based on the updated molecular inversion probe; and
obtaining a set of decision rules that are specific to determine the prevalent lineage, wherein the determining the one or more scores of the consensus sequence is determined using the set of decision rules.

8. A system comprising:
one or more data processors; and
a non-transitory computer readable medium storing instructions which, when executed on the one or more data processors, cause the one or more data processors to perform:
obtaining nucleic acid from a sample that was obtained from a subject;
capturing a target molecule in the nucleic acid using a molecular inversion probe under hybridization conditions;
amplifying the target molecule using polymerase chain reaction (PCR) to obtain a plurality of amplified molecules;
for each molecule in the plurality of amplified molecules,
ligating an adapter to each end of the molecule to create a circular molecule; and
sequencing the circular molecule to obtain sequence reads;
generating, using a computing system, a sequencing file comprising the sequence reads of each molecule in the plurality of amplified molecules and a position of each sequence read in a reference genome of a virus by aligning the sequence read to the reference genome of the virus; and
generating, using the computing system and the sequencing file, a reporting file for the subject, wherein the reporting file comprises a predicted lineage of the virus in the sample, wherein the generating comprises:
generating a consensus sequence for the target molecule based on sequence reads of each molecule in the plurality of amplified molecules, wherein a nucleotide identity is assigned to a position in the consensus sequence if at least a predetermined number of the sequence reads has the nucleotide identity in the position, and wherein an “N” is assigned to a position in the consensus sequence if less than a predetermined number of the sequence reads has the nucleotide identity in the position;
determining one or more scores of the consensus sequence based on the reference genome of the virus or a library of the virus, wherein the one or more scores are determined based on a distribution of mutations of the virus; and
determining the predicted lineage of the virus in the sample based on the one or more scores of the consensus sequence.

9. The system of claim 8, wherein the reporting file further comprises a presence or absence of the virus in the sample, and wherein the presence or absence of the virus in the sample is determined using real-time PCR (RT-PCR) or based on the one or more scores of the consensus sequence.

10. The system of claim 8, wherein the virus is monkeypox (MPX) virus.

11. The system of claim 8, wherein the molecular inversion probe consists of two binding sites about 600-700 bp apart.

12. The system of claim 8, wherein the sequencing file is a Binary Alignment Map (BAM) file, and the reporting file is a Variant Call Format (VCF) file.

13. The system of claim 8, wherein the one or more data processors are caused to further perform providing a treatment plan or clinical testing protocol for the subject, wherein the treatment plan comprises administering antiviral medications for the subject.

14. The system of claim 8, wherein the one or more data processors are caused to further perform:
obtaining a prevalent lineage of the virus for a subject population, wherein the prevalent lineage is determined based on the predicted lineage of the virus in the sample;
updating the molecular inversion probes to capture the target molecule, wherein the target molecule is specific to the prevalent lineage;
updating the adapters based on the updated molecular inversion probe; and
obtaining a set of decision rules that are specific to determine the prevalent lineage, wherein the determining the one or more scores of the consensus sequence is determined using the set of decision rules.

15. A computer-program product tangibly embodied in a non-transitory machine-readable medium, including instructions configured to cause one or more data processors to perform:
obtaining nucleic acid from a sample that was obtained from a subject;
capturing a target molecule in the nucleic acid using a molecular inversion probe under hybridization conditions;
amplifying the target molecule using polymerase chain reaction (PCR) to obtain a plurality of amplified molecules;
for each molecule in the plurality of amplified molecules,
ligating an adapter to each end of the molecule to create a circular molecule; and
sequencing the circular molecule to obtain sequence reads;
generating, using a computing system, a sequencing file comprising the sequence reads of each molecule in the plurality of amplified molecules and a position of each sequence read in a reference genome of a virus by aligning the sequence read to the reference genome of the virus; and
generating, using the computing system and the sequencing file, a reporting file for the subject, wherein the reporting file comprises a predicted lineage of the virus in the sample, wherein the generating comprises:
generating a consensus sequence for the target molecule based on sequence reads of each molecule in the plurality of amplified molecules, wherein a nucleotide identity is assigned to a position in the consensus sequence if at least a predetermined number of the sequence reads has the nucleotide identity in the position, and wherein an “N” is assigned to a position in the consensus sequence if less than a predetermined number of the sequence reads has the nucleotide identity in the position;
determining one or more scores of the consensus sequence based on the reference genome of the virus or a library of the virus, wherein the one or more scores are determined based on a distribution of mutations of the virus; and
determining the predicted lineage of the virus in the sample based on the one or more scores of the consensus sequence.

16. The computer-program product of claim 15, wherein the reporting file further comprises a presence or absence of the virus in the sample, and wherein the presence or absence of the virus in the sample is determined using real-time PCR (RT-PCR) or based on the one or more scores of the consensus sequence.

17. The computer-program product of claim 15, wherein the molecular inversion probe consists of two binding sites about 600-700 bp apart.

18. The computer-program product of claim 15, wherein the sequencing file is a Binary Alignment Map (BAM) file, and the reporting file is a Variant Call Format (VCF) file.

19. The computer-program product of claim 15, wherein the one or more data processors are caused to further perform providing a treatment plan or clinical testing protocol for the subject, wherein the treatment plan comprises administering antiviral medications for the subject.

20. The computer-program product of claim 15, wherein the one or more data processors are caused to further perform:
obtaining a prevalent lineage of the virus for a subject population, wherein the prevalent lineage is determined based on the predicted lineage of the virus in the sample;
updating the molecular inversion probes to capture the target molecule, wherein the target molecule is specific to the prevalent lineage;
updating the adapters based on the updated molecular inversion probe; and
obtaining a set of decision rules that are specific to determine the prevalent lineage, wherein the determining the one or more scores of the consensus sequence is determined using the set of decision rules.
